(19) United States
(12) Patent Application Publication
(10) Pub. No.: US 2012/0295965 A1
(43) Pub. Date: Nov. 22, 2012

(54) FUSED THIOPHENES AS DUAL INHIBITORS OF EGFR/VEGFR AND THEIR USE IN THE TREATMENT OF CANCER

(76) Inventors: Huayun Deng, Painted Post, NY (US); Ye Fang, Painted Post, NY (US); Ann MeeJin Ferrie, Painted Post, NY (US); Mingqian He, Horseheads, NY (US); Weijun Niu, Painted Post, NY (US); Haiyan Sun, Chandler, AZ (US); Elizabeth Tran, Painted Post, NY (US); Ying Wei, Painted Post, NY (US)

(21) Appl. No.: 13/519,431

(22) PCT Filed: Dec. 16, 2010
(86) PCT No.: PCT/US10/60663

§ 371 Date: Jun. 27, 2012

Related U.S. Application Data
(60) Provisional application No. 61/291,747, filed on Dec. 31, 2009.

(51) Int. Cl. A61K 31/381; G01N 21/55; A61P 35/00; C07D 495/04; C07D 495/14

(52) U.S. Cl. 514/443; 549/50; 549/43; 435/7.21

(57) ABSTRACT

Disclosed are compositions and methods related to identification of modulators of EGFR and VEGFR.

### CROSS-REFERENCE TO RELATED APPLICATIONS

This application claims the benefit of priority under 35 U.S.C. §119(e) of U.S. Provisional Application Ser. No. 61/291,747 filed on Dec. 31, 2009.

### BACKGROUND

Despite many therapeutic successes, cancer is the second-most frequent cause of death in the United States and is set to become the most common in the relatively near future. Cancer comes in many different forms—both anatomically and molecularly—and whereas in several of these (for example, some leukemias, lymphomas, testicular and pediatric cancer) drug therapy can markedly increase survival, in many of the common adult epithelial tumors the impact is modest at best. The overall success with oncology drug development in recent years has been mixed, even though over 30 new cancer treatments have been approved by the US FDA since 2001. Many of these approved drugs are antibodies, and others are not first-in-class agents. Eight tyrosine kinase inhibitors have been approved for clinical use, and dozens more are in late-stage development.

Furthermore, attrition rates for oncology drugs in the clinic are worse than for other disease areas: figures for 1990-2000 show a 5% success rate in the clinic, compared with 11% overall. Moreover, failure often occurs very late in the clinical development process. Reasons for the failure of candidate drugs for cancer and other diseases have been identified. In the 1990s, poor pharmacokinetics and bioavailability predominated in late stage failures. Technical solutions (involving predictive assays to triage compounds with permeability and metabolic liabilities) were implemented to address this, and by 2000 failure from this cause had fallen from 40% to 10%. The main causes of attrition are now insufficient therapeutic activity (30%) and toxicity (30%). These risks can be reduced by identifying better predictive and molecularly defined animal models of cancers and in vitro models of mechanism-based and off-target toxicity. However, drugs acting on new molecular targets are inherently risky. Risk can be minimized by selecting only the best targets, and by using biomarkers to identify the most appropriate subjects and to demonstrate proof of concept for the intended mechanism of action. Disclosed are methods and compositions for identifying improved pharmaceuticals, and the identification of molecules having unique properties making them better drugs and drug candidates.

### SUMMARY

Disclosed herein are compounds which modulate EGFR (epidermal growth factor receptor, including EGFR and her2/neu).

Disclosed herein are compounds which modulate VEGFR (vascular endothelial growth factor/vascular permeability factor receptor, including Flt1 and KDR/Flk1).

Disclosed herein are compounds which modulate EGFR and VEGFR.

Disclosed are compounds having structural formula (I) and (II) or a pharmaceutically acceptable sale, solvate, clathrate, or prodrug thereof, wherein R¹, R², R³, R⁴, R⁵, R⁶, R⁷ and R⁸ are defined herein.

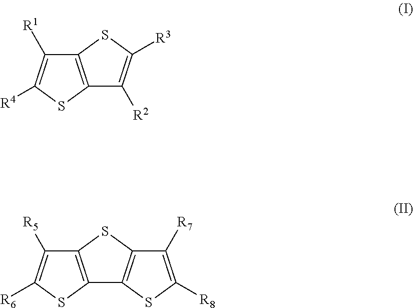

These compounds can be useful as therapeutic agents for modulating both EGFR (epidermal growth factor receptor) and VEGFR (vascular endothelial growth factor/vascular permeability factor receptor, including Flt1 and KDR/Flk1), and for anti-cancer therapy and wherein EGFR and VEGFR-1 are contributed to cancer cell growth and metastasis, and antiangiogenic therapy, respectively.

Disclosed herein are methods using label-free cellular indexing approach to screen EGFR, VEGFR, and dual EGFR and VEGFR inhibitors.

### BRIEF DESCRIPTION OF FIGURES

FIG. 1 shows the Epidermal growth factor receptor tyrosine kinase activity in the absence (“kinase”) and the presence of different compounds, wherein all compounds were assayed at 25 micromolar, except for the two control compounds (staurosporine and Iressa) whose concentrations were indicated in the graph.

FIG. 2 shows the FLT1 (VEGFR1) tyrosine kinase activity in the absence (“kinase”) and the presence of different compounds, wherein all compounds were assayed at 25 micromolar, except for the two control compounds (staurosporine and Iressa) whose concentrations were indicated in the graph.

FIG. 3 shows the KDR/Flk1 (VEGFR2) tyrosine kinase activity in the absence (“kinase”) and the presence of different compounds, wherein all compounds were assayed at 25 micromolar, except for the two control compounds (staurosporine and Iressa) whose concentrations were indicated in the graph.

FIG. 4A-4F shows a specific panel of cells/markers-based biosensor cellular indexing method to determine the compounds to be dual EGFR and VEGFR inhibitors. The cell panel consists of at least two types of cells wherein each cell expresses at least one of the two receptors. At least two markers, one is the agonist for one of the two receptors, and another one is an activator that transactivates another one of the two receptors, are chosen. (A-F). The DMR modulation index of the known VEGFR2 inhibitor I (A); the known VEGFR2 inhibitor II (B); the known EGFR inhibitor lavendustin A (C); the known potent EGFR inhibitor A1478 (D); the known Flt3 selective inhibitor III (E); and the known PDK1/AKT/Flt1 dual pathway inhibitor (F), respectively. The modulation index were generated against 4 markers across two distinct cell lines—the EGFR agonist EGF in A431 (EGF at 32 nM), and the EGFRs agonist EGF in HT29 (EGF at 2 nM), the hERG activator mallotoxin (MTX) in HT29 (MTX at 16 micromlar), and the NTS1/NTS3 agonist neurotensin (NT) in HT29 (NT at 2 nM). The EGF responses in A431 cells include the early P-DMR event (˜5 min after EGF stimulation) and the subsequent N-DMR event (˜30 min after EGF stimulation). The EGF responses in HT29 include the early P-DMR event (˜5 min after EGF stimulation) and the late P-DMR event (˜50 min after EGF stimulation), whereas the mallotoxin response in HT29 is the P-DMR response 50 min after MTX stimulation, and the NT response in HT29 is the P-DMR 50 min after NT stimulation. In all cases, the amplitudes of respective DMR events were used as the basis to calculate the percentages of modulation by each inhibitor.

FIG. 5A-5D shows a specific panel of cells/markers-based biosensor cellular indexing method to determine the compounds to be dual EGFR and VEGFR inhibitors. (A-D). The DMR modulation index of D (A); A (B), B (C) and G (D), respectively. The modulation index were generated against 4 markers across two distinct cell lines—the EGFR agonist EGF in A431 (EGF at 32 nM), and the EGFRs agonist EGF in HT29 (EGF at 2 nM), the hERG activator mallotoxin (MTX) in HT29 (MTX at 16 micromlar), and the NTS1/NTS3 agonist neurotensin (NT) in HT29 (NT at 2 nM). The EGF responses in A431 cells include the early P-DMR event (˜5 min after EGF stimulation) and the subsequent N-DMR event (˜30 min after EGF stimulation). The EGF responses in HT29 include the early P-DMR event (˜5 min after EGF stimulation) and the late P-DMR event (˜50 min after EGF stimulation), whereas the mallotoxin response in HT29 is the P-DMR response 50 min after MTX stimulation, and the NT response in HT29 is the P-DMR 50 min after NT stimulation. In all cases, the amplitudes of respective DMR events were used as the basis to calculate the percentages of modulation by each inhibitor.

FIG. 6A-6D shows a specific panel of cells/markers-based biosensor cellular indexing method to determine the compounds to be dual EGFR and VEGFR inhibitors. (A-D). The DMR modulation index of C (A); I (B), H(C) and J (D), respectively. The modulation index were generated against 4 markers across two distinct cell lines—the EGFR agonist EGF in A431 (EGF at 32 nM), and the EGFRs agonist EGF in HT29 (EGF at 2 nM), the hERG activator mallotoxin (MTX) in HT29 (MTX at 16 micromlar), and the NTS1/NTS3 agonist neurotensin (NT) in HT29 (NT at 2 nM). The EGF responses in A431 cells include the early P-DMR event (˜5 min after EGF stimulation) and the subsequent N-DMR event (˜30 min after EGF stimulation). The EGF responses in HT29 include the early P-DMR event (˜5 min after EGF stimulation) and the late P-DMR event (˜50 min after EGF stimulation), whereas the mallotoxin response in HT29 is the P-DMR response 50 min after MTX stimulation, and the NT response in HT29 is the P-DMR 50 min after NT stimulation. In all cases, the amplitudes of respective DMR events were used as the basis to calculate the percentages of modulation by each inhibitor.

FIG. 7A-7D shows a specific panel of cells/markers-based biosensor cellular indexing method to determine the compounds to be dual EGFR and VEGFR inhibitors. (A-D). The DMR modulation index of K (A); L (B), E (C) and F (D), respectively. The modulation index were generated against 4 markers across two distinct cell lines—the EGFR agonist EGF in A431 (EGF at 32 nM), and the EGFRs agonist EGF in HT29 (EGF at 2 nM), the hERG activator mallotoxin (MTX) in HT29 (MTX at 16 micromlar), and the NTS1/NTS3 agonist neurotensin (NT) in HT29 (NT at 2 nM). The EGF responses in A431 cells include the early P-DMR event (˜5 min after EGF stimulation) and the subsequent N-DMR event (˜30 min after EGF stimulation). The EGF responses in HT29 include the early P-DMR event (˜5 min after EGF stimulation) and the late P-DMR event (˜50 min after EGF stimulation), whereas the mallotoxin response in HT29 is the P-DMR response 50 min after MTX stimulation, and the NT response in HT29 is the P-DMR 50 min after NT stimulation. In all cases, the amplitudes of respective DMR events were used as the basis to calculate the percentages of modulation by each inhibitor. All modulation indexes shown in FIGS. 4 to 7 were showed as the modulation percentage of each molecule against the markers in the following order: EGF for A431 (the early P-DMR, the subsequent N-DMR), EGF for HT29 (early P-DMR, and late P-DMR), mallotoxin for HT29 (the P-DMR), and NT for HT29 (the P-DMR).

### DETAIL DESCRIPTION

### A. Tyrosine Kinases and Receptor Tyrosine Kinases

Tyrosine kinases promote cell growth, survival and proliferation, and are the target of frequent oncogenic mutations in tumors. Receptor tyrosine kinases (RTKs) are important regulators of cell survival, migration, and proliferation as well as angiogenesis and their over-expression or deregulation leads to uncontrollable cellular signaling and cancer. RTKs consist of families of growth factor receptors such as the platelet-derived growth factor receptors (PDGFRs), vascular endothelial growth factor receptors (VEGFRs), EGFRs, and among several others. RTKs are transmembrane receptors consisting of an extracellular ligand binding domain, a hydrophobic transmembrane domain and a cytoplasmic domain which contains regulatory regions and the catalytic tyrosine kinase domain with binding sites for both ATP and substrate which allows for autophosphorylation—the critical step in signal transduction pathways. RTK activation often requires the formation of homodimers or heterodimers with other RTKs. Nonreceptor tyrosine kinases do not have an extracellular domain and are usually dimers. Several small molecule inhibitors of RTKs are currently in clinical trials as antitumor agents and the majority of these are targeted at the ATP binding site of tyrosine kinases.

Platelet-derived growth factor receptors PDGFRs (PDGFR-a, PDGFR-b), and vascular endothelial growth factor receptors VEGFRs (Flt1, KDR/Flk1, and Flt4) are RTK subfamilies that play key roles in tumor angiogenesis and therefore have been targeted for the development of anti-cancer therapies. Angiogenesis is the formation of new blood vessels from existing vasculature. Angiogenesis occurs during development and in normal adults during wound healing, pregnancy and corpus luteum formation. Angiogenesis is initiated by factors intrinsic to the tumor cells that induce migration and proliferation of endothelial cells. Angiogenesis requires the transduction of signals from the extracellular domain of endothelial cells to the nucleus which are, either directly or indirectly, receptor mediated and some of the receptors are receptor tyrosine kinases (RTK).

Human disease states associated with angiogenesis include retinopathies, endometriosis, psoriasis, atherosclerosis, rheumatoid arthritis and the growth and metastasis of tumors. Angiogenesis plays a pivotal role in the growth of solid tumors and their invasion and metastasis. Angiogenesis is a prerequisite for tumor growth as well as metastatic spread and describes the recruitment of blood vessels by a growing primary tumor or metastasis. Inhibition of tumor angiogenesis has thus provided an attractive target for the development of antiangiogenesis agents as antitumor agents. Antiangiogenic therapy is targeted to non-tumor cells (endothelial cells) which are expected to have less ability to mutate in order to produce resistance compared with tumor cells. Thus, antiangiogenesis agents have afforded new paradigms for the treatment of cancer. Antiangiogenetic strategies, e.g., neutralizing antibodies directed against VEGF, have already been proven successful in experimental thyroid cancer.

1. VEGFRs

Vascular endothelial growth factor (VEGF) is an important signaling protein involved in both vasculogenesis (the formation of the embryonic circulatory system) and angiogenesis. VEGF activity is restricted mainly to cells of the vascular endothelium, although it does have effects on a limited number of other cell types (e.g. stimulation monocyte/macrophage migration). In vitro, VEGF has been shown to stimulate endothelial cell mitogenesis and cell migration. VEGF also enhances microvascular permeability and is sometimes referred to as vascular permeability factor. VEGF-A binds to VEGFR-1 (Flt-1) and VEGFR-2 (KDR/Flk-1). VEGFR-2 appears to mediate almost all of the known cellular responses to VEGF. The function of VEGFR-1 is less well defined, although it is thought to modulate VEGFR-2 signaling. Another function of VEGFR-1 may be to act as a dummy/decoy receptor, sequestering VEGF from VEGFR-2 binding (this appears to be particularly important during vasculogenesis in the embryo). A third receptor has been discovered (VEGFR-3), however, VEGF-A is not a ligand for this receptor. VEGFR-3 mediates lymphangiogenesis in response to VEGF-C and VEGF-D.

VEGFR-2 is the principal receptor that mediates VEGF stimulation in angiogenesis. The receptors for VEGF are almost exclusively expressed on endothelial cells. Targeted inhibition or disruption of VEGFR-2 results in abrogation of angiogenesis and tumor growth. In addition, VEGF and VEGFRs are overexpressed in many tumor types. Several inhibitors of VEGFR-2 have provided antitumor activity. Notable among these are the pyrroloindolinones and quinazolines exemplified by SU5416 which has been in clinical trials and ZD6474 which is currently in clinical trials as an antitumor agent. Recent reports indicate that inhibition of VEGFR-1 (Flt-1) could be a therapeutic target not only for tumor angiogenesis but also for the inflammation associated with tumors. Thus VEGFR-1 is also a viable target against cancer.

2. HER Family

The human epidermal growth factor receptor (HER) family members include EGFR (erbB1), HER2/neu (erbB2), HER3 (erbB3), and HER4 (erbB4) that are structurally related, and all except HER3 contain intracellular tyrosine kinase domain. All of the HER members, except HER2, bind to extracellular ligands. Activation of EGFR and HER2/neu induces a cascade of downstream signaling through several pathways, such as mitogen-activated protein kinase (MAPK) and PI3-kinase/Akt/mTOR, resulting in cellular proliferation, differentiation, survival, motility, adhesion, and repair. EGFR and HER2/neu are overexpressed or abnormally activated in several epithelial malignancies. This finding eventually led to the United States Food and Drug Administration's (FDA) approval of several agents specifically targeting these receptors. These include monoclonal antibodies such as cetuximab and panitumumab for colorectal cancers, trastuzumab for breast cancers, and small-molecule inhibitors, such as erlotinib, for lung and pancreatic cancers. These anticancer drugs are now readily available to the general oncology community, and reviews of their clinical development have been published. Research in this area currently focuses on targeting more than one HER-family receptor simultaneously. Lapatinib, a small-molecule inhibitor, is such an agent that targets both EGFR and HER2/neu receptors, and was approved by the US FDA for the treatment of breast cancer. Other drugs that target more than one HER-family receptor and that are under clinical development include BMS-599626, PF-00299804, and BMS-690514.

3. ATP Binding Site of RTK

The ATP-binding site of RTKs has been shown to be a viable target for rational drug design. Of these, the most successful have been those based on the quinazoline, indoline, pyrido[d]pyrimidines scaffolds which are ATP-competitive.

Single RTK inhibition by small molecules is a possible mechanism of cancer therapy. Simultaneous targeting of two or more RTKs represents a novel approach for (antiangiogenic) therapy of tumors. These RTKs are present on endothelial cells (VEGFR, PDGFR), in tumor cells (FGFR, PDGFR) and pericytes (FGFR, PDGFR). Thus simultaneous inhibition of more than one RTK could provide synergistic effects against tumors. For example, sorafenib (BAY43-9006) is an oral, dual inhibitor of Raf and vascular endothelial growth factor receptor (VEGFR). The molecule has demonstrated preclinical antineoplastic activity against a wide spectrum of human cancers. It has potent in vitro inhibitory effects against Raf-1, B-Raf, VEGFR-2, platelet-derived growth factor receptor (PDGFR), and VEGFR-3.

### B. Multitargeted Agents

Multitargeted agents represent the next generation of targeted therapies in solid tumors. The benefits of individually targeting the vascular endothelial growth factor receptor (VEGFR) and epidermal growth factor receptor (EGFR) signaling pathways have been clinically validated in recent years in a number of solid tumor types including non-small cell lung cancer (NSCLC). Given the heterogeneity of this tumor type and potential crosstalk between these key signaling pathways (which are known to play a critical role in tumor growth, metastasis, and angiogenesis), dual inhibition of the VEGFR and EGFR signaling pathways has the potential to offer additional clinical benefits in NSCLC. A number of approaches to inhibiting both VEGFR and EGFR signaling are currently under investigation, including monotherapy with a multitargeted tyrosine kinase inhibitor (e.g., vandetanib, AEE788, XL647, BMS-690514) or a combination of single-targeted therapies (e.g., bevacizumab, cetuximab, erlotinib, gefitinib). For example, vandetanib is a novel, orally available inhibitor of different intracellular signaling pathways involved in tumor growth, progression, and angiogenesis: vascular endothelial growth factor receptor-2, epidermal growth factor receptor, and RET oncogenic rearrangement during Transfection tyrosine kinase activity. Preclinical and early clinical data (phase I and II trials) support combined inhibition of the VEGFR and EGFR pathways in NSCLC, and also medullary thyroid cancer. Overall, combined inhibition strategies are well tolerated and have shown promise in early clinical studies.

### C. Compounds

Disclosed herein are compounds of structural formula (I) or (II) or a pharmaceutically acceptable salt, solvate, clathrate, or prodrug thereof, wherein R¹, R², R³, R⁴, R⁵, R⁶, R⁷ and R⁸ are defined herein. These compounds are useful as therapeutic agents for modulating EGFR and VEGFR tyrosine kinase activities, and for improved prevention and treatment of EGFR and/or VEGFR associated diseases such as cancers.

Disclosed herein are compounds that relate to thieno[3,2-β]-thiophene and derivatives, as described in formula (I), and formula (II), and pharmaceutically acceptable salts, solvates, clathrates, and prodrugs thereof,

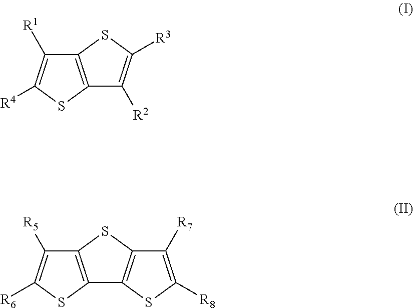

wherein R¹, R², R⁴, R⁵, R⁶ and R⁷ is independently selected from —H, alkyl, alkynyl, alkenyl, aryl, alkylaryl, cycloalkyl, cyclo aklenyl, heterocycle, cyclohexyl, amino, ester, aldehyde, hydroxyl, alkoxy, thiol group, thioalkyl group, halogen, halide, or an acyl halide. Preferred groups are described in, for example, US patent applications US20070265418 and US20070161776 each of which is herein incorporated by reference in their entirities at least for material related to functional groups.

R³ and R⁸ are independently selected from —COOH, aldehyde or ester.

Disclosed herein are compounds that relate to thieno[3,2-β]-thiophene and derivatives, as described in formula (III), and formula (IV), and pharmaceutically acceptable salts, solvates, clathrates, and prodrugs thereof,

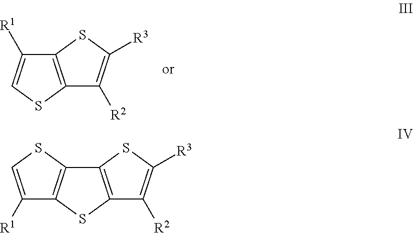

wherein:
R¹ is halogen, hydrogen, or unsubstituted C₁₋₂₀ alkyl,
R² is hydrogen or unsubstituted C₁₋₂₀ alkyl;
R³ is carboxyl;
at least one of R¹ and R² is unsubstituted C₁₋₂₀ alkyl; and the compound is a dual EGFR and VEGFR inhibitor.

Disclosed are uses of thieno[3,2-β]-thiophene as a scaffold to build chemicals for dual EGFR and VEGFR inhibitors. The building blocks can be added onto the thieno[3,2-β]-thiophene to form new chemicals for dual EGFR and VEGFR inhibitors. These building blocks can be chosen from fragments and functional groups known to be important for interactions or binding to either EGFR or VEGFR or both.

Disclosed are compounds or a pharmaceutically acceptable salt, solvate, clathrate, or prodrug thereof that can inhibit the activity of both EGFR and VEGFR tyrosine kinases. In particular, a compound disclosed herein or a pharmaceutically acceptable salt, solvate, clathrate, or prodrug thereof can inhibit the deregulated activity of EGFR and/or VEGFR tyrosine kinases, thus improve the prevention and treatment of EGFR or VEGFR associated human diseases such as cancers.

Disclosed are pharmaceutical compositions comprising an effective amount of a compound of the invention or a pharmaceutically accepted salt, solvate, clathrate, or prodrug thereof; and a pharmaceutically acceptable carrier or vehicle. These compositions may further comprise additional agents. These compositions are useful for inhibiting the deregulated activity of EGFR and/or VEGFR tyrosine kinases, thus improve the prevention and treatment of EGFR or VEGFR associated human diseases such as cancers.

### D. Methods of Treating

Disclosed are methods for treating or preventing EGFR or VEGFR associated human diseases such as cancers, comprising administering to a subject in need thereof a compound of the invention or a pharmaceutically acceptable salt, solvate, clathrate, or prodrug thereof, or a pharmaceutical composition comprising a compound of the invention or a pharmaceutically acceptable salt, solvate, clathrate, or prodrug thereof. These methods may also comprise administering to the subject an additional agent separately or in a combination composition with the compound of the invention or a pharmaceutically acceptable salt, solvate, clathrate, or prodrug thereof.

Disclosed are methods for treating EGFR or VEGFR associated human diseases such as cancers in vivo or in vitro using an effective amount of a compound of the invention, or a pharmaceutically acceptable salt, solvate, clathrate or prodrug thereof, or a pharmaceutical composition comprising an effective amount of a compound of the invention or a pharmaceutically acceptable salt, solvate, clathrate or prodrug thereof.

All disclosed methods can be practiced with the disclosed compounds alone, or in combination with other agents, or other anticancer drugs.

Also disclosed are methods where the subjects have been identified as needing treatment for an EGFR or VEGFR related disease, as well as methods where the subjects are monitored for the disease progression or regression after administration of a compound or composition as described herein. Also included are methods including a step wherein the treatment of the subject, the administration of a compound or composition as described herein, is adjusted or maintained because of the state of the disease as tested after one or more administrations as described herein.

Also disclosed are methods to optimize therapeutic efficacy for treatment of an EGFR or VEGFR related disease comprising administering a drug providing a dual EGFR/VEGFR inhibitor to a subject having said EGFR or VEGFR related disease, and determining the level of a marker for the EGFR or VEGFR related disease in said subject having said EGFR or VEGFR related disease, wherein the level of a marker for the EGFR or VEGFR related disease effects the amount of dual EGFR/VEGFR inhibitor subsequently administered. The marker comprises a tumor marker. The level of the marker less than the level of the marker at an earlier time indicates that the amount of said drug subsequently administered to said subject does not need to be increased. The EGFR or VEGFR related disease is selected from the group consisting of skin cancer, colorectal cancer, breast cancer, thyroid cancer, non-small cell lung cancer, lung cancer, or pancreatic cancer. The level of marker for the EGFR or VEGFR related disease is often determined in a blood sample, using an analytical method, such as mass spectrometry, high pressure liquid chromatography, and an antibody based assay. Alternatively, the marker can be a toxicity marker to measure the level of a liver enzyme or blood enzyme.

### E. Methods of Screening

Disclosed are methods to screening dual EGFR and VEGFR inhibitors. The methods are related to label-free cellular indexing approaches, particularly using a specific panel of cells/markers-based biosensor cellular indexing method to determine the compounds to be dual EGFR and VEGFR inhibitors. The method of screening dual EGFR and VEGFR inhibitors, comprising the steps: a) selecting at least two distinct cell lines each expressing at least one receptor of EGFR or VEGFR; b) selecting at least two markers, wherein one marker is the agonist for one of the two receptors, and another marker is an activator that transactivates another one of the two receptors; c) incubating each marker in the absence and presence of a test compound to its respective cell line; d) analyzing the biosensor signal of each marker in the absence and presence of a test compound on the marker respective cell line with a label free biosensor assay; e) analyzing the effect of the test compound on all marker induced biosensor signals; f) determining if the test compound is a dual EGFR and VEGFR. The marker is selected from an agonist for EGFR, a G protein-coupled receptor agonist that transactivates EGFR, an agonist for VEGFR, or a hERG activator that transactivates VEGFR. The marker is preferably assayed at a concentration being close to its EC80 or EC100 to trigger its biosensor signal in its respective cells.

The cell panel preferably consists of at least two types of cells wherein each cell expresses at least one of the two receptors. At least two markers, one is the agonist for one of the two receptors, and another one is an activator that transactivates another one of the two receptors, are chosen; both can be for one cell type, or different cell types, within the cell panel. Specific examples are human skin carcinoma cell line A431, and human colon carcinoma cell line HT29. A431 endogenously overexpresses EGFR, whose hyperactivation is linked to the human skin cancer. HT29 endogenously expresses both EGFR (erbB1) and her2/neu. HT29 also expresses functional FLT1 (VEGFR1) receptor, but not other VEGFRs including KDR/Flk1 and FLT3 (M. Calvani, et al., Cancer Res. 2008, 68: 285). HT29 also endogenously expresses both hERG ion channels and neurotensin receptor NTS1. The label-free cellular assay modulation index is generated for any test compound against 4 markers across the two distinct cell lines—the EGFR agonist EGF in A431 (EGF at 32 nM), and the EGFRs agonist EGF in HT29 (EGF at 2 nM), the hERG activator mallotoxin (MTX) in HT29 (MTX at 16 micromlar), and the NTS1/NTS3 agonist neurotensin (NT) in HT29 (NT at 2 nM). The modulation index is calculated based on the percentages of modulation of each marker-induced biosensor signal by each inhibitor. The biosensor signal used for calculation of the index includes, but not limited to, both the early P-DMR event (˜5 min after EGF stimulation) and the subsequent N-DMR event (˜30 min after EGF stimulation) for the EGF responses in A431 cells, both the early P-DMR event (˜5 min after EGF stimulation) and the late P-DMR event (˜50 min after EGF stimulation) for the EGF responses in HT29, the P-DMR response 50 min after MTX stimulation for the mallotoxin response in HT29, and the P-DMR 50 min after NT stimulation (the NT response in HT29). In all cases, the amplitudes of respective DMR events are used as the basis to calculate the percentages of modulation by each inhibitor.

### F. Compounds 2

Disclosed herein are compositions and methods for modulating EGFR or VEGFR in a subject, comprising administering one or more compounds chosen from:

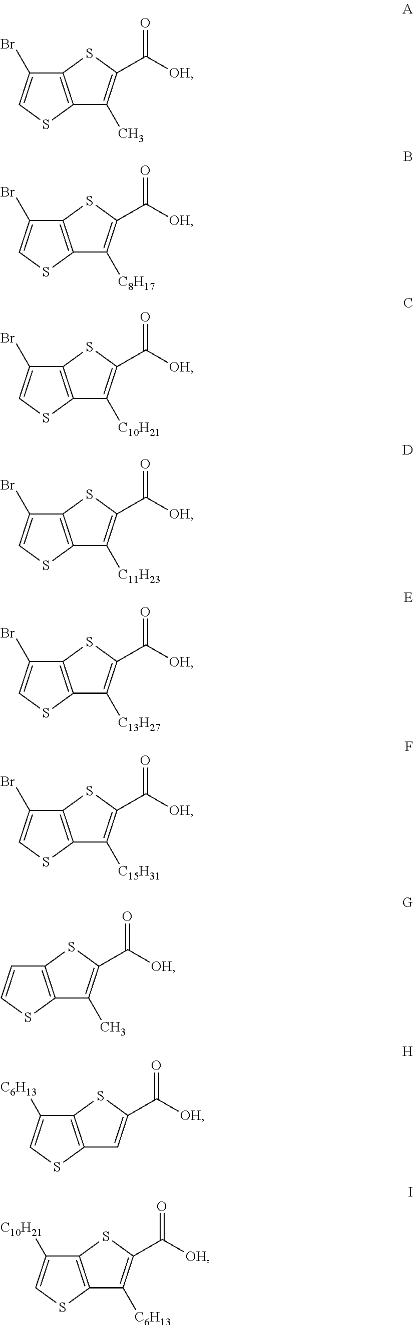

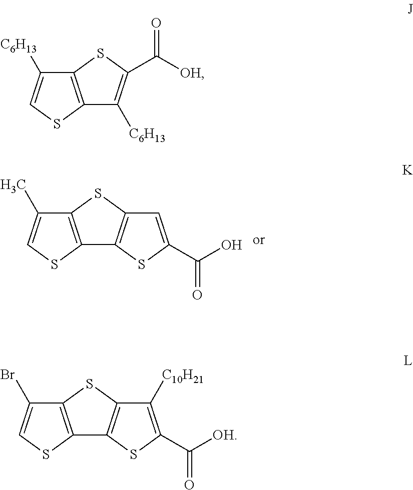

### G. Biosensors

1. Acoustic Biosensors

Acoustic biosensors such as quartz crystal resonators utilize acoustic waves to characterize cellular responses. The acoustic waves are generally generated and received using piezoelectric. An acoustic biosensor is often designed to operate in a resonant type sensor configuration. In a typical setup, thin quartz discs are sandwiched between two gold electrodes. Application of an AC signal across electrodes leads to the excitation and oscillation of the crystal, which acts as a sensitive oscillator circuit. The output sensor signals are the resonance frequency and motional resistance. The resonance frequency is largely a linear function of total mass of adsorbed materials when the biosensor surface is rigid. Under liquid environments the acoustic sensor response is sensitive not only to the mass of bound molecules, but also to changes in viscoelastic properties and charge of the molecular complexes formed or live cells. By measuring the resonance frequency and the motion resistance of cells associated with the crystals, cellular processes including cell adhesion and cytotoxicity can be studied in real time.

2. Electrical Biosensors

Electrical biosensors employ impedance to characterize cellular responses including cell adhesion. In a typical setup, live cells are brought in contact with a biosensor surface wherein an integrated electrode array is embedded. A small AC pulse at a constant voltage and high frequency is used to generate an electric field between the electrodes, which are impeded by the presence of cells. The electric pulses are generated on site using the integrated electric circuit; and the electrical current through the circuit is followed with time. The resultant impedance is a measure of changes in the electrical conductivity of the cell layer. The cellular plasma membrane acts as an insulating agent forcing the current to flow between or beneath the cells, leading to quite robust changes in impedance. Impedance-based measurements have been applied to study a wide range of cellular events, including cell adhesion and spreading, cell micromotion, cell morphological changes, and cell death, and cell signaling.

3. Optical Biosensors

Optical biosensors primarily employ a surface-bound electromagnetic wave to characterize cellular responses. The surface-bound waves can be achieved either on gold substrates using either light excited surface plasmons (surface plasmon resonance, SPR) or on dielectric substrate using diffraction grating coupled waveguide mode resonances (resonance waveguide grating, RWG). For SPR, the readout is the resonance angle at which a minimal in intensity of reflected light occurs. Similarly, for RWG biosensor, the readout is the resonance angle or wavelength at which a maximum incoupling efficiency is achieved. The resonance angle or wavelength is a function of the local refractive index at or near the sensor surface. Unlike SPR, which is limited to a few of flow channels for assaying, RWG biosensors are amenable for high throughput screening (HTS) and cellular assays, due to recent advancements in instrumentation and assays. In a typical RWG, the cells are directly placed into a well of a microtiter plate in which a biosensor consisting of a material with high refractive index is embedded. Local changes in the refractive index lead to a dynamic mass redistribution (DMR) signal of live cells upon stimulation. These biosensors have been used to study diverse cellular processes including receptor biology, ligand pharmacology, and cell adhesion.

The present invention preferably uses resonant waveguide grating biosensors, such as Corning Epic® systems. Epic® system includes the commercially available wavelength integration system, or angular interrogation system or swept wavelength imaging system (Corning Inc., Corning, N.Y.). The commercial system consists of a temperature-control unit, an optical detection unit, with an on-board liquid handling unit with robotics, or an external liquid accessory system with robotics. The detection unit is centered on integrated fiber optics, and enables kinetic measures of cellular responses with a time interval of ˜7 or 15 sec. The compound solutions were introduced by using either the on-board liquid handling unit, or the external liquid accessory system; both of which use conventional liquid handling system.

4. Biosensors and Biosensor Assays

Label-free cell-based assays generally employ a biosensor to monitor molecule-induced responses in living cells. The molecule can be naturally occurring or synthetic, and can be a purified or unpurified mixture. A biosensor typically utilizes a transducer such as an optical, electrical, calorimetric, acoustic, magnetic, or like transducer, to convert a molecular recognition event or a molecule-induced change in cells contacted with the biosensor into a quantifiable signal. These label-free biosensors can be used for molecular interaction analysis, which involves characterizing how molecular complexes form and disassociate over time, or for cellular response, which involves characterizing how cells respond to stimulation. The biosensors that are applicable to the present methods can include, for example, optical biosensor systems such as surface plasmon resonance (SPR) and resonant waveguide grating (RWG) biosensors, resonant mirrors, ellipsometers, and electric biosensor systems such as bioimpedance systems.

i. SPR Biosensors and Systems

SPR relies on a prism to direct a wedge of polarized light, covering a range of incident angles, into a planar glass substrate bearing an electrically conducting metallic film (e.g., gold) to excite surface plasmons. The resultant evanescent wave interacts with, and is absorbed by, free electron clouds in the gold layer, generating electron charge density waves (i.e., surface plasmons) and causing a reduction in the intensity of the reflected light. The resonance angle at which this intensity minimum occurs is a function of the refractive index of the solution close to the gold layer on the opposing face of the sensor surface

ii. RWG Biosensors and Systems

An RWG biosensor can include, for example, a substrate (e.g., glass), a waveguide thin film with an embedded grating or periodic structure, and a cell layer. The RWG biosensor utilizes the resonant coupling of light into a waveguide by means of a diffraction grating, leading to total internal reflection at the solution-surface interface, which in turn creates an electromagnetic field at the interface. This electromagnetic field is evanescent in nature, meaning that it decays exponentially from the sensor surface; the distance at which it decays to 1/e of its initial value is known as the penetration depth and is a function of the design of a particular RWG biosensor, but is typically on the order of about 200 nm. This type of biosensor exploits such evanescent wave to characterize ligand-induced alterations of a cell layer at or near the sensor surface.

RWG instruments can be subdivided into systems based on angle-shift or wavelength-shift measurements. In a wavelength-shift measurement, polarized light covering a range of incident wavelengths with a constant angle is used to illuminate the waveguide; light at specific wavelengths is coupled into and propagates along the waveguide. Alternatively, in angle-shift instruments, the sensor is illuminated with monochromatic light and the angle at which the light is resonantly coupled is measured.

The resonance conditions are influenced by the cell layer (e.g., cell confluency, adhesion and status), which is in direct contact with the surface of the biosensor. When a ligand or an analyte interacts with a cellular target (e.g., a GPCR, an ion channel, a kinase) in living cells, any change in local refractive index within the cell layer can be detected as a shift in resonant angle (or wavelength).

The Corning® Epic® system uses RWG biosensors for label-free biochemical or cell-based assays (Corning Inc., Corning, N.Y.). The Epic® System consists of an RWG plate reader and SBS (Society for Biomolecular Screening) standard microtiter plates. The detector system in the plate reader exploits integrated fiber optics to measure the shift in wavelength of the incident light, as a result of ligand-induced changes in the cells. A series of illumination-detection heads are arranged in a linear fashion, so that reflection spectra are collected simultaneously from each well within a column of a 384-well microplate. The whole plate is scanned so that each sensor can be addressed multiple times, and each column is addressed in sequence. The wavelengths of the incident light are collected and used for analysis. A temperature-controlling unit can be included in the instrument to minimize spurious shifts in the incident wavelength due to the temperature fluctuations. The measured response represents an averaged response of a population of cells. Varying features of the systems can be automated, such as sample loading, and can be multiplexed, such as with a 96 or 386 well microtiter plate. Liquid handling is carried out by either on-board liquid handler, or an external liquid handling accessory. Specifically, molecule solutions are directly added or pipetted into the wells of a cell assay plate having cells cultured in the bottom of each well. The cell assay plate contains certain volume of assay buffer solution covering the cells. A simple mixing step by pipetting up and down certain times can also be incorporated into the molecule addition step.

iii. Electrical Biosensors and Systems

Electrical biosensors consist of a substrate (e.g., plastic), an electrode, and a cell layer. In this electrical detection method, cells are cultured on small gold electrodes arrayed onto a substrate, and the system's electrical impedance is followed with time. The impedance is a measure of changes in the electrical conductivity of the cell layer. Typically, a small constant voltage at a fixed frequency or varied frequencies is applied to the electrode or electrode array, and the electrical current through the circuit is monitored over time. The ligand-induced change in electrical current provides a measure of cell response. Impedance measurement for whole cell sensing was first realized in 1984. Since then, impedance-based measurements have been applied to study a wide range of cellular events, including cell adhesion and spreading, cell micromotion, cell morphological changes, and cell death. Classical impedance systems suffer from high assay variability due to use of a small detection electrode and a large reference electrode. To overcome this variability, the latest generation of systems, such as the CellKey system (MDS Sciex, South San Francisco, Calif.) and RT-CES (ACEA Biosciences Inc., San Diego, Calif.), utilize an integrated circuit having a microelectrode array.

iv. High Spatial Resolution Biosensor Imaging Systems

Optical biosensor imaging systems, including SPR imaging systems, ellipsometry imaging systems, and RWG imaging systems, offer high spatial resolution, and can be used in embodiments of the disclosure. For example, SPR Imager®II (GWC Technologies Inc) uses prism-coupled SPR, and takes SPR measurements at a fixed angle of incidence, and collects the reflected light with a CCD camera. Changes on the surface are recorded as reflectivity changes. Thus, SPR imaging collects measurements for all elements of an array simultaneously.

A swept wavelength optical interrogation system based on RWG biosensor for imaging-based application can be employed. In this system, a fast tunable laser source is used to illuminate a sensor or an array of RWG biosensors in a microplate format. The sensor spectrum can be constructed by detecting the optical power reflected from the sensor as a function of time as the laser wavelength scans, and analysis of the measured data with computerized resonant wavelength interrogation modeling results in the construction of spatially resolved images of biosensors having immobilized receptors or a cell layer. The use of an image sensor naturally leads to an imaging based interrogation scheme. 2 dimensional label-free images can be obtained without moving parts.

Alternatively, angular interrogation system with transverse magnetic or p-polarized TM₀ mode can also be used. This system consists of a launch system for generating an array of light beams such that each illuminates a RWG sensor with a dimension of approximately 200 μm×3000 μm or 200 μm×2000 μm, and a CCD camera-based receive system for recording changes in the angles of the light beams reflected from these sensors. The arrayed light beams are obtained by means of a beam splitter in combination with diffractive optical lenses. This system allows up to 49 sensors (in a 7×7 well sensor array) to be simultaneously sampled at every 3 seconds, or up to the whole 384well microplate to be simultaneously sampled at every 10 seconds.

Alternatively, a scanning wavelength interrogation system can also be used. In this system, a polarized light covering a range of incident wavelengths with a constant angle is used to illuminate and scan across a waveguide grating biosensor, and the reflected light at each location can be recorded simultaneously. Through scanning, a high resolution image across a biosensor can also be achieved

v. Dynamic Mass Redistribution (DMR) Signals in Living Cells

The cellular response to stimulation through a cellular target can be encoded by the spatial and temporal dynamics of downstream signaling networks. For this reason, monitoring the integration of cell signaling in real time can provide physiologically relevant information that is useful in understanding cell biology and physiology.

Optical biosensors including resonant waveguide grating (RWG) biosensors, can detect an integrated cellular response related to dynamic redistribution of cellular matters, thus providing a non-invasive means for studying cell signaling. All optical biosensors are common in that they can measure changes in local refractive index at or very near the sensor surface. In principle, almost all optical biosensors are applicable for cell sensing, as they can employ an evanescent wave to characterize ligand-induced change in cells. The evanescent-wave is an electromagnetic field, created by the total internal reflection of light at a solution-surface interface, which typically extends a short distance (hundreds of nanometers) into the solution at a characteristic depth known as the penetration depth or sensing volume.

Recently, theoretical and mathematical models have been developed that describe the parameters and nature of optical signals measured in living cells in response to stimulation with ligands. These models, based on a 3-layer waveguide system in combination with known cellular biophysics, link the ligand-induced optical signals to specific cellular processes mediated through a receptor.

Because biosensors measure the average response of the cells located at the area illuminated by the incident light, a highly confluent layer of cells can be used to achieve optimal assay results. Due to the large dimension of the cells as compared to the short penetration depth of a biosensor, the sensor configuration is considered as a non-conventional three-layer system: a substrate, a waveguide film with a grating structure, and a cell layer. Thus, a ligand-induced change in effective refractive index (i.e., the detected signal) can be, to first order, directly proportional to the change in refractive index of the bottom portion of the cell layer:

ΔN=S(C)Δn_{c}

where S(C) is the sensitivity to the cell layer, and Δn_{c} the ligand-induced change in local refractive index of the cell layer sensed by the biosensor. Because the refractive index of a given volume within a cell is largely determined by the concentrations of bio-molecules such as proteins, Δn_{c} can be assumed to be directly proportional to ligand-induced change in local concentrations of cellular targets or molecular assemblies within the sensing volume. Considering the exponentially decaying nature of the evanescent wave extending away from the sensor surface, the ligand-induced optical signal is governed by:

$$Δ N = S ( C ) α d {\sum }_{i}^{ } Δ {C}_{i} [ {e}^{\frac{- {z}_{i}}{Δ {Z}_{C}}} - {e}^{\frac{- {z}_{i + 1}}{Δ {Z}_{C}}} ]$$

where ΔZ_{c} is the penetration depth into the cell layer, α the specific refraction increment (about 0.18/mL/g for proteins), zᵢ the distance where the mass redistribution occurs, and d an imaginary thickness of a slice within the cell layer. Here the cell layer is divided into an equal-spaced slice in the vertical direction. The equation above indicates that the ligand-induced optical signal is a sum of mass redistribution occurring at distinct distances away from the sensor surface, each with an unequal contribution to the overall response. Furthermore, the detected signal, in terms of wavelength or angular shifts, is primarily sensitive to mass redistribution occurring perpendicular to the sensor surface. Because of its dynamic nature, it also is referred to as dynamic mass redistribution (DMR) signal.

5. Cells and Biosensors

Cells rely on multiple cellular pathways or machineries to process, encode and integrate the information they receive. Unlike the affinity analysis with optical biosensors that specifically measures the binding of analytes to a protein target, living cells are much more complex and dynamic.

To study cell signaling, cells can be brought into contact with the surface of a biosensor, which can be achieved through cell culture. These cultured cells can be attached onto the biosensor surface through three types of contacts: focal contacts, close contacts and extracellular matrix contacts, each with its own characteristic separation distance from the surface. As a result, the basal cell membranes are generally located away from the surface by ˜10-100 nm. For suspension cells, the cells can be brought in contact with the biosensor surface through either covalent coupling of cell surface receptors, or specific binding of cell surface receptors, or simple settlement or sedimentation by gravity force. For this reason, biosensors are able to sense the bottom portion of cells.

Cells, in many cases, exhibit surface-dependent adhesion and proliferation. In order to achieve robust cell assays, the biosensor surface can require a coating to enhance cell adhesion and proliferation. However, the surface properties can have a direct impact on cell biology. For example, surface-bound ligands can influence the response of cells, as can the mechanical compliance of a substrate material, which dictates how it will deform under forces applied by the cell. Due to differing culture conditions (time, serum concentration, confluency, etc.), the cellular status obtained can be distinct from one surface to another, and from one condition to another. Thus, special efforts to control cellular status can be necessary in order to develop biosensor-based cell assays.

Cells are dynamic objects with relatively large dimensions—typically in the range of tens of microns. Even without stimulation, cells constantly undergo micromotion—a dynamic movement and remodeling of cellular structure, as observed in tissue culture by time lapse microscopy at the sub-cellular resolution, as well as by bio-impedance measurements at the nanometer level.

Under un-stimulated conditions cells generally produce an almost net-zero DMR response as examined with a RWG biosensor. This is partly because of the low spatial resolution of optical biosensors, as determined by the large size of the laser spot and the long propagation length of the coupled light. The size of the laser spot determines the size of the area studied—and usually only one analysis point can be tracked at a time. Thus, the biosensor typically measures an averaged response of a large population of cells located at the light incident area. Although cells undergo micromotion at the single cell level, the large populations of cells give rise to an average net-zero DMR response. Furthermore, intracellular macromolecules are highly organized and spatially restricted to appropriate sites in mammalian cells. The tightly controlled localization of proteins on and within cells determines specific cell functions and responses because the localization allows cells to regulate the specificity and efficiency of proteins interacting with their proper partners and to spatially separate protein activation and deactivation mechanisms. Because of this control, under un-stimulated conditions, the local mass density of cells within the sensing volume can reach an equilibrium state, thus leading to a net-zero optical response. In order to achieve a consistent optical response, the cells examined can be cultured under conventional culture conditions for a period of time such that most of the cells have just completed a single cycle of division.

Living cells have exquisite abilities to sense and respond to exogenous signals. Cell signaling was previously thought to function via linear routes where an environmental cue would trigger a linear chain of reactions resulting in a single well-defined response. However, research has shown that cellular responses to external stimuli are much more complicated. It has become apparent that the information that cells receive can be processed and encoded into complex temporal and spatial patterns of phosphorylation and topological relocation of signaling proteins. The spatial and temporal targeting of proteins to appropriate sites can be crucial to regulating the specificity and efficiency of protein-protein interactions, thus dictating the timing and intensity of cell signaling and responses. Pivotal cellular decisions, such as cytoskeletal reorganization, cell cycle checkpoints and apoptosis, depend on the precise temporal control and relative spatial distribution of activated signal-transducers. Thus, cell signaling mediated through a cellular target such as G protein-coupled receptor (GPCR) typically proceeds in an orderly and regulated manner, and consists of a series of spatial and temporal events, many of which lead to changes in local mass density or redistribution in local cellular matters of cells. These changes or redistribution, when occurring within the sensing volume, can be followed directly in real time using optical biosensors

6. DMR Signal is a Physiological Response of Living Cells

Through comparison with conventional pharmacological approaches for studying receptor biology, it has been shown that when a ligand is specific to a receptor expressed in a cell system, the ligand-induced DMR signal is receptor-specific, dose-dependent and saturate-able. For a great number of G protein-coupled receptor (GPCR) ligands, the efficacies (measured by EC₅₀ values) are found to be almost identical to those measured using conventional methods. In addition, the DMR signals exhibit expected desensitization patterns, as desensitization and re-sensitization is common to all GPCRs. Furthermore, the DMR signal also maintains the fidelity of GPCR ligands, similar to those obtained using conventional technologies. In addition, the biosensor can distinguish full agonists, partial agonists, inverse agonists, antagonists, and allosteric modulators. Taken together, these findings indicate that the DMR is capable of monitoring physiological responses of living cells.

7. DMR Signals Contain Systems Cell Biology Information of Ligand-Receptor Pairs in Living Cells

The stimulation of cells with a ligand leads to a series of spatial and temporal events, non-limiting examples of which include ligand binding, receptor activation, protein recruitment, receptor internalization and recycling, second messenger alternation, cytoskeletal remodeling, gene expression, and cell adhesion changes. Because each cellular event has its own characteristics (e.g., kinetics, duration, amplitude, mass movement), and the biosensor is primarily sensitive to cellular events that involve mass redistribution within the sensing volume, these cellular events can contribute differently to the overall DMR signal. Chemical biology, cell biology and biophysical approaches can be used to elucidate the cellular mechanisms for a ligand-induced DMR signal. Recently, chemical biology, which directly uses chemicals for intervention in a specific cell signaling component, has been used to address biological questions. This is possible due to the identification of a great number of modulators that specifically control the activities of many different types of cellular targets. This approach has been adopted to map the signaling and its network interactions mediated through a receptor, including epidermal growth factor (EGF) receptor, and G_{q} and Gₛ-coupled receptors.

EGFR belongs to the family of receptor tyrosine kinases. EGF binds to and stimulates the intrinsic protein-tyrosine kinase activity of EGFR, initiating a signal transduction cascade, principally involving the MAPK, Akt and JNK pathways. Upon EGF stimulation, there are many events leading to mass redistribution in A431 cells—a cell line endogenously over-expressing EGFRs. It is known that EGFR signaling depends on cellular status. As a result, the EGF-induced DMR signals are also dependent on the cellular status. In quiescent cells obtained through 20 hr culturing in 0.1% fetal bovine serum, EGF stimulation leads to a DMR signal with three distinct and sequential phases: (i) a positive phase with increased signal (P-DMR), (ii) a transition phase, and (iii) a decay phase (N-DMR). Chemical biology and cell biology studies show that the EGF-induced DMR signal is primarily linked to the Ras/MAPK pathway, which proceeds through MEK and leads to cell detachment. Two lines of evidence indicate that the P-DMR is mainly due to the recruitment of intracellular targets to the activated receptors at the cell surface. First, blockage of either dynamin or clathrin activity has little effect on the amplitude of the P-DMR event. Dynamin and clathrin, two downstream components of EGFR activation, play crucial roles in executing EGFR internalization and signaling. Second, the blockage of MEK activity partially attenuates the P-DMR event. MEK is an important component in the MAPK pathway, which first translocates from the cytoplasm to the cell membrane, followed by internalization with the receptors, after EGF stimulation.

On the other hand, the N-DMR event is due to cell detachment and receptor internalization. Fluorescent images show that EGF stimulation leads to a significant number of receptors internalized and cell detachment. It is known that blockage of either receptor internalization or MEK activity prevents cell detachment, and receptor internalization requires both dynamin and clathrin. This indicates that blockage of either dynamin or clathrin activity should inhibit both receptor internalization and cell detachment, while blockage of MEK activity should only inhibit cell detachment, but not receptor internalization. As expected, either dynamin or clathrin inhibitors completely inhibit the EGF-induced N-DMR (˜100%), while MEK inhibitors only partially attenuate the N-DMR (˜80%). Fluorescent images also confirm that blocking the activity of dynamin, but not MEK, impairs the receptor internalization

8. DMR Signals Contain Systems Cell Pharmacology Information of a Ligand Acting on Living Cells.

Since the DMR signal is an integrated cellular response consisting of contributions of many cellular events involving dynamic redistribution of cellular matters within the bottom portion of cells, a ligand-induced biosensor signal, such as a DMR signal contains systems cell pharmacology information. It is known that GPCRs often display rich behaviors in cells, and that many ligands can induce operative bias to favor specific portions of the cell machinery and exhibit pathway-biased efficacies. Thus, it is highly possibly that a ligand can have multiple efficacies, depending on how cellular events downstream of the receptor are measured and used as readout(s) for the ligand pharmacology. It is difficult in practice for conventional cell assays, which are mostly pathway-biased and assay only a single signaling event, to systematically represent the signaling potentials of GPCR ligands. However, because label-free biosensors cellular assays do not require prior knowledge of cell signaling, and are pathway-unbiased and pathway-sensitive, these biosensor cellular assays are amenable to studying ligand-selective signaling as well as systems cell pharmacology of any ligands.

9. Biosensor Parameters

A label-free biosensor such as RWG biosensor or bioimpedance biosensor is able to follow in real time ligand-induced cellular response. The non-invasive and manipulation-free biosensor cellular assays do not require prior knowledge of cell signaling. The resultant biosensor signal contains high information relating to receptor signaling and ligand pharmacology. Multi-parameters can be extracted from the kinetic biosensor response of cells upon stimulation. These parameters include, but not limited to, the overall dynamics, phases, signal amplitudes, as well as kinetic parameters including the transition time from one phase to another, and the kinetics of each phase (see Fang, Y., and Ferrie, A. M. (2008) “label-free optical biosensor for ligand-directed functional selectivity acting on β2 adrenoceptor in living cells”. FEBS Lett. 582, 558-564; Fang, Y., et al., (2005) “Characteristics of dynamic mass redistribution of EGF receptor signaling in living cells measured with label free optical biosensors”. Anal. Chem., 77, 5720-5725; Fang, Y., et al., (2006) “Resonant waveguide grating biosensor for living cell sensing”. Biophys. J., 91, 1925-1940).

10. Method and Composition/Compound Relationships

The methods disclosed herein, as well as the compositions and compounds which can be used in the methods, can arise from a number of different classes, such as materials, substance, molecules, and ligands. Also disclosed is a specific subset of these classes, unique to label free biosensor assays, called markers, for example, EGF as a marker for EGFR activation.

It is understood that mixtures of these classes, such as a molecule mixture are also disclosed and can be used in the disclosed methods.

In certain methods, unknown molecules, test molecules, drug candidate molecules as well as known molecules can be used.

In certain methods or situations, modulating or modulators play a role. Likewise, known modulators can be used.

In certain methods, as well as compositions, cells are involved, and cells can undergo culturing and cell cultures can be used as discussed herein.

The methods disclosed herein involve assays that use biosensors. In certain assays, they are performed in either an agonist or antagonist mode. Often the assays involve treating cells with one or more classes, such as a material, a substance, or a molecule. It is also understood that subjects can be treated as well, as discussed herein.

In certain methods, contacting between a molecule, for example, and a cell can take place. In the disclosed methods, responses, such as cellular response, which can be manifested as a biosensor response, such as a DMR response, can be detected. These and other responses can be assayed. In certain methods the signals from a biosensor can be robust biosensor signals or robust DMR signals.

The disclosed methods utilizing label free biosensors can produce profiles, such as primary profiles, secondary profiles, and modulation profiles. These profiles and others can be used for making determinations about molecules, for example, and can be used with any of the classes discussed herein.

Also disclosed are libraries and panels of compounds or compositions, such as molecules, cells, materials, or substances disclosed herein. Also disclosed are specific panels, such as marker panels and cell panels.

The disclosed methods can utilize a variety of aspects, such as biosensor signals, DMR signals, normalizing, controls, positive controls, modulation comparisons, indexes, biosensor indexes, DMR indexes, molecule biosensor indexes, molecule DMR indexes, molecule indexes, modulator biosensor indexes, modulator DMR indexes, molecule modulation indexes, known modulator biosensor indexes, known modulator DMR indexes, marker biosensor indexes, marker DMR indexes, modulating the biosensor signal of a marker, modulating the DMR signal, potentiating, and similarity of indexes.

Any of the compositions, compounds, or anything else disclosed herein can be characterized in any way disclosed herein.

Disclosed are methods that rely on characterizations, such as potentiate and inhibit and like words.

In certain methods, receptors or cellular targets are used. Certain methods can provide information about signaling pathway(s) as well as molecule-treated cells and other cellular processes.

In certain embodiments, a certain potency or efficacy becomes a characteristic, and the direct action (of a drug candidate molecule, for example) can be assayed.

The disclosed methods can be performed on or with samples.

The disclosed methods and compositions and compounds can be involved in optimizing, for example, for therapeutic efficacy or toxicity, as discuss herein. For example, optimization can occurs using markers, such as a disease or toxicity marker, and for example, the methods disclosed herein can all utilize an analytical method or methods.

### H. Definitions

Various embodiments of the disclosure will be described in detail with reference to drawings, if any. Reference to various embodiments does not limit the scope of the disclosure, which is limited only by the scope of the claims attached hereto. Additionally, any examples set forth in this specification are not intended to be limiting and merely set forth some of the many possible embodiments for the claimed invention.

1. A

As used in the specification and the appended claims, the singular forms “a,” “an” and “the” or like terms include plural referents unless the context clearly dictates otherwise. Thus, for example, reference to “a pharmaceutical carrier” includes mixtures of two or more such carriers, and the like.

2. Abbreviations

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, “M” for molar, and like abbreviations).

3. About

About modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

4. Assaying

Assaying, assay, or like terms refers to an analysis to determine a characteristic of a substance, such as a molecule or a cell, such as for example, the presence, absence, quantity, extent, kinetics, dynamics, or type of an a cell's optical or bioimpedance response upon stimulation with one or more exogenous stimuli, such as a ligand or marker. Producing a biosensor signal of a cell's response to a stimulus can be an assay.

5. Assaying the Response

“Assaying the response” or like terms means using a means to characterize the response. For example, if a molecule is brought into contact with a cell, a biosensor can be used to assay the response of the cell upon exposure to the molecule.

6. Agonism and Antagonism Mode

The agonism mode or like terms is the assay wherein the cells are exposed to a molecule to determine the ability of the molecule to trigger biosensor signals such as DMR signals, while the antagonism mode is the assay wherein the cells are exposed to a maker in the presence of a molecule to determine the ability of the molecule to modulate the biosensor signal of cells responding to the marker.

7. Biosensor

Biosensor or like terms refer to a device for the detection of an analyte that combines a biological component with a physicochemical detector component. The biosensor typically consists of three parts: a biological component or element (such as tissue, microorganism, pathogen, cells, or combinations thereof), a detector element (works in a physicochemical way such as optical, piezoelectric, electrochemical, thermometric, or magnetic), and a transducer associated with both components. The biological component or element can be, for example, a living cell, a pathogen, or combinations thereof. In embodiments, an optical biosensor can comprise an optical transducer for converting a molecular recognition or molecular stimulation event in a living cell, a pathogen, or combinations thereof into a quantifiable signal.

8. Biosensor Response

A “biosensor response”, “biosensor output signal”, “biosensor signal” or like terms is any reaction of a sensor system having a cell to a cellular response. A biosensor converts a cellular response to a quantifiable sensor response. A biosensor response is an optical response upon stimulation as measured by an optical biosensor such as RWG or SPR or it is a bioimpedence response of the cells upon stimulation as measured by an electric biosensor. Since a biosensor response is directly associated with the cellular response upon stimulation, the biosensor response and the cellular response can be used interchangeably, in embodiments of disclosure.

9. Biosensor Signal

A “biosensor signal” or like terms refers to the signal of cells measured with a biosensor that is produced by the response of a cell upon stimulation.

10. Cell

Cell or like term refers to a small usually microscopic mass of protoplasm bounded externally by a semipermeable membrane, optionally including one or more nuclei and various other organelles, capable alone or interacting with other like masses of performing all the fundamental functions of life, and forming the smallest structural unit of living matter capable of functioning independently including synthetic cell constructs, cell model systems, and like artificial cellular systems.

A cell can include different cell types, such as a cell associated with a specific disease, a type of cell from a specific origin, a type of cell associated with a specific target, or a type of cell associated with a specific physiological function. A cell can also be a native cell, an engineered cell, a transformed cell, an immortalized cell, a primary cell, an embryonic stem cell, an adult stem cell, a cancer stem cell, or a stem cell derived cell.

Human consists of about 210 known distinct cell types. The numbers of types of cells can almost unlimited, considering how the cells are prepared (e.g., engineered, transformed, immortalized, or freshly isolated from a human body) and where the cells are obtained (e.g., human bodies of different ages or different disease stages, etc).

11. Cell Culture

“Cell culture” or “cell culturing” refers to the process by which either prokaryotic or eukaryotic cells are grown under controlled conditions. “Cell culture” not only refers to the culturing of cells derived from multicellular eukaryotes, especially animal cells, but also the culturing of complex tissues and organs.

12. Cell Panel

A “cell panel” or like terms is a panel which comprises at least two types of cells. The cells can be of any type or combination disclosed herein.

13. Cellular Response

A “cellular response” or like terms is any reaction by the cell to a stimulation.

14. Cellular Process

A cellular process or like terms is a process that takes place in or by a cell. Examples of cellular process include, but not limited to, proliferation, apoptosis, necrosis, differentiation, cell signal transduction, polarity change, migration, or transformation.

15. Cellular Target

A “cellular target” or like terms is a biopolymer such as a protein or nucleic acid whose activity can be modified by an external stimulus. Cellular targets are most commonly proteins such as enzymes, kinases, ion channels, and receptors.

16. Characterizing

Characterizing or like terms refers to gathering information about any property of a substance, such as a ligand, molecule, marker, or cell, such as obtaining a profile for the ligand, molecule, marker, or cell.

17. Comprise

Throughout the description and claims of this specification, the word “comprise” and variations of the word, such as “comprising” and “comprises,” means “including but not limited to,” and is not intended to exclude, for example, other additives, components, integers or steps.

18. Consisting Essentially of

“Consisting essentially of” in embodiments refers, for example, to a surface composition, a method of making or using a surface composition, formulation, or composition on the surface of the biosensor, and articles, devices, or apparatus of the disclosure, and can include the components or steps listed in the claim, plus other components or steps that do not materially affect the basic and novel properties of the compositions, articles, apparatus, and methods of making and use of the disclosure, such as particular reactants, particular additives or ingredients, a particular agents, a particular cell or cell line, a particular surface modifier or condition, a particular ligand candidate, or like structure, material, or process variable selected. Items that may materially affect the basic properties of the components or steps of the disclosure or may impart undesirable characteristics to the present disclosure include, for example, decreased affinity of the cell for the biosensor surface, aberrant affinity of a stimulus for a cell surface receptor or for an intracellular receptor, anomalous or contrary cell activity in response to a ligand candidate or like stimulus, and like characteristics.

19. Components

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these molecules may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

20. Contacting

Contacting or like terms means bringing into proximity such that a molecular interaction can take place, if a molecular interaction is possible between at least two things, such as molecules, cells, markers, at least a compound or composition, or at least two compositions, or any of these with an article(s) or with a machine. For example, contacting refers to bringing at least two compositions, molecules, articles, or things into contact, i.e. such that they are in proximity to mix or touch. For example, having a solution of composition A and cultured cell B and pouring solution of composition A over cultured cell B would be bringing solution of composition A in contact with cell culture B. Contacting a cell with a ligand would be bringing a ligand to the cell to ensure the cell have access to the ligand.

It is understood that anything disclosed herein can be brought into contact with anything else. For example, a cell can be brought into contact with a marker or a molecule, a biosensor, and so forth.

21. Compounds and Compositions

Compounds and compositions have their standard meaning in the art. It is understood that wherever, a particular designation, such as a molecule, substance, marker, cell, or reagent compositions comprising, consisting of, and consisting essentially of these designations are disclosed. Thus, where the particular designation marker is used, it is understood that also disclosed would be compositions comprising that marker, consisting of that marker, or consisting essentially of that marker. Where appropriate wherever a particular designation is made, it is understood that the compound of that designation is also disclosed. For example, if particular biological material, such as EGF, is disclosed EGF in its compound form is also disclosed.

22. Control

The terms control or “control levels” or “control cells” or like terms are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard. For example, a control can refer to the results from an experiment in which the subjects or objects or reagents etc are treated as in a parallel experiment except for omission of the procedure or agent or variable etc under test and which is used as a standard of comparison in judging experimental effects. Thus, the control can be used to determine the effects related to the procedure or agent or variable etc. For example, if the effect of a test molecule on a cell was in question, one could a) simply record the characteristics of the cell in the presence of the molecule, b) perform a and then also record the effects of adding a control molecule with a known activity or lack of activity, or a control composition (e.g., the assay buffer solution (the vehicle)) and then compare effects of the test molecule to the control. In certain circumstances once a control is performed the control can be used as a standard, in which the control experiment does not have to be performed again and in other circumstances the control experiment should be run in parallel each time a comparison will be made.

23. Chemistry Terms

i. alkyl

The term “alkyl” as used herein is a branched or unbranched saturated hydrocarbon moiety. “Unbranched” or “Branched” alkyls comprise a non-cyclic, saturated, straight or branched chain hydrocarbon moiety having from 1 to 24 carbons, 1 to 20 carbons, 1 to 15 carbons, 1 to 12 carbons, 1 to 8 carbons, 1 to 6 carbons, or 1 to 4 carbon atoms. It is understood that the term “alkyl” also encompass straight or branched chain hydrocarbon moiety having 1, 2, 3, 4, 5, 6, 7, 8, 9, 10, 11, 12, 13, 14, 15, 16, 17, 18, 19, 20, 21, 22, 23, or 24 carbon atoms. Examples of such alkyl radicals include methyl, ethyl, propyl, butyl, pentyl, hexyl, heptyl, octyl, n-propyl, iso-propyl, butyl, n-butyl, sec-butyl, t-butyl, amyl, t-amyl, n-pentyl and the like. Lower alkyls comprise a noncyclic, saturated, straight or branched chain hydrocarbon residue having from 1 to 4 carbon atoms, i.e., C₁-C₄ alkyl.

Moreover, the term “alkyl” as used throughout the specification and claims is intended to include both “unsubstituted alkyls” and “substituted alkyls”, the later denotes an alkyl radical analogous to the above definition that is further substituted with one, two, or more additional organic or inorganic substituent groups. Suitable substituent groups include but are not limited to H, alkyl, alkenyl, alkynyl, hydroxyl, cycloalkyl, heterocyclyl, amino, mono-substituted amino, di-substituted amino, unsubstituted or substituted amido, carbonyl, halogen, sulfhydryl, sulfonyl, sulfonato, sulfamoyl, sulfonamide, azido, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl. It will be understood by those skilled in the art that an “alkoxy” can be a substituted of a carbonyl substituted “alkyl” forming an ester. When more than one substituent group is present then they can be the same or different. The organic substituent moieties can comprise from 1 to 12 carbon atoms, or from 1 to 6 carbon atoms, or from 1 to 4 carbon atoms. It will be understood by those skilled in the art that the moieties substituted on the “alkyl” chain can themselves be substituted, as described above, if appropriate.

ii. Alkenyl

The term “alkenyl” as used herein is an alkyl residue as defined above that also comprises at least one carbon-carbon double bond in the backbone of the hydrocarbon chain. Examples include but are not limited to vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2-heptenyl, 3-heptenyl, 4-heptenyl, 5-heptenyl, 6-heptenyl and the like. The term “alkenyl” includes dienes and trienes of straight and branch chains.

iii. alkynyl

The term “alkynyl” as used herein is an alkyl residue as defined above that comprises at least one carbon-carbon triple bond in the backbone of the hydrocarbon chain. Examples include but are not limited ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

iv. cycloalkyl

The term “cycloalkyl” as used herein is a saturated hydrocarbon structure wherein the structure is closed to form at least one ring. Cycloalkyls typically comprise a cyclic radical containing 3 to 8 ring carbons, such as cyclopropyl, cyclobutyl, cyclopentyl, cyclopenyl, cyclohexyl, cycloheptyl and the like. Cycloalkyl radicals can be multicyclic and can contain a total of 3 to 18 carbons, or preferably 4 to 12 carbons, or 5 to 8 carbons. Examples of multicyclic cycloalkyls include decahydronapthyl, adamantyl, and like radicals.

Moreover, the term “cycloalkyl” as used throughout the specification and claims is intended to include both “unsubstituted cycloalkyls” and “substituted cycloalkyls”, the later denotes an cycloalkyl radical analogous to the above definition that is further substituted with one, two, or more additional organic or inorganic substituent groups that can include but are not limited to hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl. When the cycloalkyl is substituted with more than one substituent group, they can be the same or different. The organic substituent groups can comprise from 1 to 12 carbon atoms, or from 1 to 6 carbon atoms, or from 1 to 4 carbon atoms.

v. cycloalkenyl

The term “cycloalkenyl” as used herein is a cycloalkyl radical as defined above that comprises at least one carbon-carbon double bond. Examples include but are not limited to cyclopropenyl, 1-cyclobutenyl, 2-cyclobutenyl, 1-cyclopentenyl, 2-cyclopentenyl, 3-cyclopentenyl, 1-cyclohexyl, 2-cyclohexyl, 3-cyclohexyl and the like.

vi. alkoxy

The term “alkoxy” as used herein is an alkyl residue, as defined above, bonded directly to an oxygen atom, which is then bonded to another moiety. Examples include methoxy, ethoxy, n-propoxy, iso-propoxy, n-butoxy, t-butoxy, iso-butoxy and the like.

vii. amino

The term “amino” as used herein is a moiety comprising a N radical substituted with zero, one or two organic substituent groups, which include but are not limited to alkyls, alkyls, cycloalkyls, aryls, or arylalkyls. If there are two substituent groups they can be different or the same. Examples of amino groups include, —NH₂, methylamino (—NH—CH₃); ethylamino (—NHCH₂CH₃), hydroxyethylamino (—NH—CH₂CH₂OH), dimethylamino, methylethylamino, diethylamino, and the like.

viii. Mono-Substituted Amino

The term “mono-substituted amino” as used herein is a moiety comprising an NH radical substituted with one organic substituent group, which include but are not limited to alkyls, substituted alkyls, cycloalkyls, aryls, or arylalkyls. Examples of mono-substituted amino groups include methylamino (—NH—CH₃); ethylamino (—NHCH₂CH₃), hydroxyEthylamino (—NH—CH₂CH₂OH), and the like.

ix. Di-Substituted Aminio

The term “di-substituted amino” as used herein is a moiety comprising a nitrogen atom substituted with two organic radicals that can be the same or different, which can be selected from but are not limited to aryl, substituted aryl, alkyl, substituted alkyl or arylalkyl, wherein the terms have the same definitions found throughout. Some examples include dimethylamino, methylethylamino, diethylamino and the like.

x. azide

As used herein, the term “azide”, “azido” and their variants refer to any moiety or compound comprising the monovalent group —N₃ or the monovalent ion —N₃.

xi. haloalkyl

The term “haloalkyl” as used herein an alkyl residue as defined above, substituted with one or more halogens, preferably fluorine, such as a trifluoromethyl, pentafluoroethyl and the like.

xii. Haloalkoxy

The term “haloalkoxy” as used herein a haloalkyl residue as defined above that is directly attached to an oxygen to form trifluoromethoxy, pentafluoroethoxy and the like.

xiii. Acyl

The term “acyl” as used herein is a R—C(O)— residue having an R group containing 1 to 8 carbons. The term “acyl” encompass acyl halide, R—(O)-halogen. Examples include but are not limited to formyl, acetyl, propionyl, butanoyl, iso-butanoyl, pentanoyl, hexanoyl, heptanoyl, benzoyl and the like, and natural or un-natural amino acids.

xiv. Acyloxy

The term “acyloxy” as used herein is an acyl radical as defined above directly attached to an oxygen to form an R—C(O)O— residue. Examples include but are not limited to acetyloxy, propionyloxy, butanoyloxy, iso-butanoyloxy, benzoyloxy and the like.

xv. Aryl

The term “aryl” as used herein is a ring radical containing 6 to 18 carbons, or preferably 6 to 12 carbons, comprising at least one aromatic residue therein. Examples of such aryl radicals include phenyl, naphthyl, and ischroman radicals. Moreover, the term “aryl” as used throughout the specification and claims is intended to include both “unsubstituted alkyls” and “substituted alkyls”, the later denotes an aryl ring radical as defined above that is substituted with one or more, preferably 1, 2, or 3 organic or inorganic substituent groups, which include but are not limited to a halogen, alkyl, alkenyl, alkynyl, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, ring wherein the terms are defined herein. The organic substituent groups can comprise from 1 to 12 carbon atoms, or from 1 to 6 carbon atoms, or from 1 to 4 carbon atoms. An aryl moiety with 1, 2, or 3 alkyl substituent groups can be referred to as “arylalkyl.” It will be understood by those skilled in the art that the moieties substituted on the “aryl” can themselves be substituted, as described above, if appropriate.

xvi. Heteroaryl

The term “heteroaryl” as used herein is an aryl ring radical as defined above, wherein at least one of the ring carbons, or preferably 1, 2, or 3 carbons of the aryl aromatic ring has been replaced with a heteroatom, which include but are not limited to nitrogen, oxygen, and sulfur atoms. Examples of heteroaryl residues include pyridyl, bipyridyl, furanyl, and thiofuranyl residues. Substituted “heteroaryl” residues can have one or more organic or inorganic substituent groups, or preferably 1, 2, or 3 such groups, as referred to herein-above for aryl groups, bound to the carbon atoms of the heteroaromatic rings. The organic substituent groups can comprise from 1 to 12 carbon atoms, or from 1 to 6 carbon atoms, or from 1 to 4 carbon atoms.

xvii. Heterocyclyl

The term “heterocyclyl” or “heterocyclic group” as used herein is a non-aromatic mono- or multi ring radical structure having 3 to 16 members, preferably 4 to 10 members, in which at least one ring structure include 1 to 4 heteroatoms (e.g. O, N, S, P, and the like). Heterocyclyl groups include, for example, pyrrolidine, oxolane, thiolane, imidazole, oxazole, piperidine, piperizine, morpholine, lactones, lactams, such as azetidiones, and pyrrolidiones, sultams, sultones, and the like. Moreover, the term “heterocyclyl” as used throughout the specification and claims is intended to include both “unsubstituted alkyls” and “substituted alkyls”, the later denotes an aryl ring radical as defined above that is substituted with one or more, preferably 1, 2, or 3 organic or inorganic substituent groups, which include but are not limited to a halogen, alkyl, alkenyl, alkynyl, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, ring wherein the terms are defined herein. The organic substituent groups can comprise from 1 to 12 carbon atoms, or from 1 to 6 carbon atoms, or from 1 to 4 carbon atoms. It will be understood by those skilled in the art that the moieties substituted on the “heterocyclyl” can themselves be substituted, as described above, if appropriate.

xviii. Halogen or Halo

The term “halo” or “halogen” refers to a fluoro, chloro, bromo or iodo group.

xix. Moiety

A “moiety” is part of a molecule (or compound, or analog, etc.). A “functional group” is a specific group of atoms in a molecule. A moiety can be a functional group or can include one or functional groups.

xx. Ester

The term “ester” as used herein is represented by the formula —C(O)OA, where A can be an alkyl, halogenated alkyl, alkenyl, alkynyl, aryl, heteroaryl, cycloalkyl, cycloalkenyl, heterocycloalkyl, or heterocycloalkenyl group described above.

xxi. Carbonate Group

The term “carbonate group” as used herein is represented by the formula —OC(O)OR, where R can be hydrogen, an alkyl, alkenyl, alkynyl, aryl, aralkyl, cycloalkyl, halogenated alkyl, or heterocycloalkyl group described above.

xxii. Keto Group

The term “keto group” as used herein is represented by the formula —C(O)R, where R is an alkyl, alkenyl, alkynyl, aryl, aralkyl, cycloalkyl, halogenated alkyl, or heterocycloalkyl group described above.

xxiii. Aldehyde

The term “aldehyde” as used herein is represented by the formula —C(O)H or —R—C(O)H, wherein R can be as defined above alkyl, alkenyl, alkoxy, aryl, heteroaryl, cycloalkyl, cycloalkenyl, heterocycloalkyl, or heterocycloalkenyl group described above.

xxiv. Carboxylic Acid

The term “carboxylic acid” as used herein is represented by the formula —C(O)OH.

xxv. Carbonyl Group

The term “carbonyl group” as used herein is represented by the formula C═O.

xxvi. Ether

The term “ether” as used herein is represented by the formula AOA¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above.

xxvii. Urethane

The term “urethane” as used herein is represented by the formula —OC(O)NRR′, where R and R′ can be, independently, hydrogen, an alkyl, alkenyl, alkynyl, aryl, aralkyl, cycloalkyl, halogenated alkyl, or heterocycloalkyl group described above.

xxviii. Silyl Group

The term “silyl group” as used herein is represented by the formula —SiRR′R″, where R, R′, and R″ can be, independently, hydrogen, an alkyl, alkenyl, alkynyl, aryl, aralkyl, cycloalkyl, halogenated alkyl, alkoxy, or heterocycloalkyl group described above.

xxix. Sulfo-Oxo Group

The term “sulfo-oxo group” as used herein is represented by the formulas —S(O)₂R, —OS(O)₂R, or, —OS(O)₂OR, where R can be hydrogen, or as defined above an alkyl, alkenyl, alkynyl, aryl, aralkyl, cycloalkyl, halogenated alkyl, or heterocycloalkyl group described above.

24. Clathrate

A compound for use in the invention may form a complex such as a “clathrate”, a drug-host inclusion complex, wherein, in contrast to solvates, the drug and host are present in stoichiometric or non-stoichiometric amounts. A compound used herein can also contain two or more organic and/or inorganic components which can be in stoichiometric or non-stoichiometric amounts. The resulting complexes can be ionised, partially ionised, or non-ionised. For a review of such complexes, see J. Pharm. ScL, 64 (8), 1269-1288, by Haleblian (August 1975).

25. Detect

Detect or like terms refer to an ability of the apparatus and methods of the disclosure to discover or sense a molecule- or a marker-induced cellular response and to distinguish the sensed responses for distinct molecules.

26. Direct Action (of a Drug Candidate Molecule)

A “direct action” or like terms is a result (of a drug candidate molecule”) acting independently on a cell.

27. DMR Signal

A “DMR signal” or like terms refers to the signal of cells measured with an optical biosensor that is produced by the response of a cell upon stimulation.

28. DMR Response

A “DMR response” or like terms is a biosensor response using an optical biosensor. The DMR refers to dynamic mass redistribution or dynamic cellular matter redistribution. A P-DMR is a positive DMR response, a N-DMR is a negative DMR response, and a RP-DMR is a recovery P-DMR response.

29. Drug Candidate Molecule

A drug candidate molecule or like terms is a test molecule which is being tested for its ability to function as a drug or a pharmacophore. This molecule may be considered as a lead molecule.

30. Efficacy

Efficacy or like terms is the capacity to produce a desired size of an effect under ideal or optimal conditions. It is these conditions that distinguish efficacy from the related concept of effectiveness, which relates to change under real-life conditions. Efficacy is the relationship between receptor occupancy and the ability to initiate a response at the molecular, cellular, tissue or system level.

31. Dual EGFR and VEGFR Inhibitor

A dual EGFR and VEGFR inhibitor is a molecule that can inhibit the kinase activities of both EGFR and VEGFR. Such an inhibitor is not only an EGFR inhibitor, but also a VEGFR inhibitor.

32. Higher and Inhibit and Like Words

The terms higher, increases, elevates, or elevation or like terms or variants of these terms, refer to increases above basal levels, e.g., as compared a control. The terms low, lower, reduces, decreases or reduction or like terms or variation of these terms, refer to decreases below basal levels, e.g., as compared to a control. For example, basal levels are normal in vivo levels prior to, or in the absence of, or addition of a molecule such as an agonist or antagonist to a cell. Inhibit or forms of inhibit or like terms refers to reducing or suppressing.

33. In the Presence of the Molecule

“in the presence of the molecule” or like terms refers to the contact or exposure of the cultured cell with the molecule. The contact or exposure can be taken place before, or at the time, the stimulus is brought to contact with the cell.

34. Index

An index or like terms is a collection of data. For example, an index can be a list, table, file, or catalog that contains one or more modulation profiles. It is understood that an index can be produced from any combination of data. For example, a DMR profile can have a P-DMR, a N-DMR, and a RP-DMR. An index can be produced using the completed date of the profile, the P-DMR data, the N-DMR data, the RP-DMR data, or any point within these, or in combination of these or other data. The index is the collection of any such information. Typically, when comparing indexes, the indexes are of like data, i.e. P-DMR to P-DMR data.

i. Biosensor Index

A “biosensor index” or like terms is an index made up of a collection of biosensor data. A biosensor index can be a collection of biosensor profiles, such as primary profiles, or secondary profiles. The index can be comprised of any type of data. For example, an index of profiles could be comprised of just an N-DMR data point, it could be a P-DMR data point, or both or it could be an impedence data point. It could be all of the data points associated with the profile curve.

ii. DMR Index

A “DMR index” or like terms is a biosensor index made up of a collection of DMR data.

35. Known Molecule

A known molecule or like terms is a molecule with known pharmacological/biological/physiological/pathophysiological activity whose precise mode of action(s) may be known or unknown.

36. Known Modulator

A known modulator or like terms is a modulator where at least one of the targets is known with a known affinity. For example, a known modulator could be a PI3K inhibitor, a PKA inhibitor, a GPCR antagonist, a GPCR agonist, a RTK inhibitor, an epidermal growth factor receptor neutralizing antibody, or a phosphodiesterase inhibition, a PKC inhibitor or activator, etc.

37. Known Modulator Biosensor Index

A “known modulator biosensor index” or like terms is a modulator biosensor index produced by data collected for a known modulator. For example, a known modulator biosensor index can be made up of a profile of the known modulator acting on the panel of cells, and the modulation profile of the known modulator against the panels of markers, each panel of markers for a cell in the panel of cells.

38. Known Modulator DMR Index

A “known modulator DMR index” or like terms is a modulator DMR index produced by data collected for a known modulator. For example, a known modulator DMR index can be made up of a profile of the known modulator acting on the panel of cells, and the modulation profile of the known modulator against the panels of markers, each panel of markers for a cell in the panel of cells.

39. Ligand

A ligand or like terms is a substance or a composition or a molecule that is able to bind to and form a complex with a biomolecule to serve a biological purpose. Actual irreversible covalent binding between a ligand and its target molecule is rare in biological systems. Ligand binding to receptors alters the chemical conformation, i.e., the three dimensional shape of the receptor protein. The conformational state of a receptor protein determines the functional state of the receptor. The tendency or strength of binding is called affinity. Ligands include substrates, blockers, inhibitors, activators, and neurotransmitters. Radioligands are radioisotope labeled ligands, while fluorescent ligands are fluorescently tagged ligands; both can be considered as ligands are often used as tracers for receptor biology and biochemistry studies. Ligand and modulator are used interchangeably.

40. Library

A library or like terms is a collection. The library can be a collection of anything disclosed herein. For example, it can be a collection, of indexes, an index library; it can be a collection of profiles, a profile library; or it can be a collection of DMR indexes, a DMR index library; Also, it can be a collection of molecule, a molecule library; it can be a collection of cells, a cell library; it can be a collection of markers, a marker library; A library can be for example, random or non-random, determined or undetermined. For example, disclosed are libraries of DMR indexes or biosensor indexes of known modulators.

41. Marker

A marker or like terms is a ligand which produces a signal in a biosensor cellular assay. The signal is, must also be, characteristic of at least one specific cell signaling pathway(s) and/or at least one specific cellular process(es) mediated through at least one specific target(s). The signal can be positive, or negative, or any combinations (e.g., oscillation). An EGFR activator, such as EGF, can be a marker for A431 cells wherein EGFRs are stably expressed.

42. Marker Panel

A “marker panel” or like terms is a panel which comprises at least two markers. The markers can be for different pathways, the same pathway, different targets, or even the same targets.

43. Marker Biosensor Index

A “marker biosensor index” or like terms is a biosensor index produced by data collected for a marker. For example, a marker biosensor index can be made up of a profile of the marker acting on the panel of cells, and the modulation profile of the marker against the panels of markers, each panel of markers for a cell in the panel of cells.

44. Marker DMR Index

A “marker biosensor index” or like terms is a biosensor DMR index produced by data collected for a marker. For example, a marker DMR index can be made up of a profile of the marker acting on the panel of cells, and the modulation profile of the marker against the panels of markers, each panel of markers for a cell in the panel of cells.

45. Material

Material is the tangible part of something (chemical, biochemical, biological, or mixed) that goes into the makeup of a physical object.

46. Mimic

As used herein, “mimic” or like terms refers to performing one or more of the functions of a reference object. For example, a molecule mimic performs one or more of the functions of a molecule.

47. Modulate

To modulate, or forms thereof, means either increasing, decreasing, or maintaining a cellular activity mediated through a cellular target. It is understood that wherever one of these words is used it is also disclosed that it could be 1%, 5%, 10%, 20%, 50%, 100%, 500%, or 1000% increased from a control, or it could be 1%, 5%, 10%, 20%, 50%, or 100% decreased from a control.

48. Modulator

A modulator or like terms is a ligand that controls the activity of a cellular target. It is a signal modulating molecule binding to a cellular target, such as a target protein.

49. Modulation comparison

A “modulation comparison” or like terms is a result of normalizing a primary profile and a secondary profile.

50. Modulator Biosensor Index

A “modulator biosensor index” or like terms is a biosensor index produced by data collected for a modulator. For example, a modulator biosensor index can be made up of a profile of the modulator acting on the panel of cells, and the modulation profile of the modulator against the panels of markers, each panel of markers for a cell in the panel of cells.

51. Modulator DMR Index

A “modulator DMR index” or like terms is a DMR index produced by data collected for a modulator. For example, a modulator DMR index can be made up of a profile of the modulator acting on the panel of cells, and the modulation profile of the modulator against the panels of markers, each panel of markers for a cell in the panel of cells.

52. Modulate the Biosensor Signal of a Marker

Modulate the biosensor signal or like terms is to cause changes of the biosensor signal or profile of a cell in response to stimulation with a marker.

53. Modulate the DMR Signal

Modulate the DMR signal or like terms is to cause changes of the DMR signal or profile of a cell in response to stimulation with a marker.

54. Molecule

As used herein, the terms “molecule” or like terms refers to a biological or biochemical or chemical entity that exists in the form of a chemical molecule or molecule with a definite molecular weight. A molecule or like terms is a chemical, biochemical or biological molecule, regardless of its size.

Many molecules are of the type referred to as organic molecules (molecules containing carbon atoms, among others, connected by covalent bonds), although some molecules do not contain carbon (including simple molecular gases such as molecular oxygen and more complex molecules such as some sulfur-based polymers). The general term “molecule” includes numerous descriptive classes or groups of molecules, such as proteins, nucleic acids, carbohydrates, steroids, organic pharmaceuticals, small molecule, receptors, antibodies, and lipids. When appropriate, one or more of these more descriptive terms (many of which, such as “protein,” themselves describe overlapping groups of molecules) will be used herein because of application of the method to a subgroup of molecules, without detracting from the intent to have such molecules be representative of both the general class “molecules” and the named subclass, such as proteins. Unless specifically indicated, the word “molecule” would include the specific molecule and salts thereof, such as pharmaceutically acceptable salts.

55. Molecule Mixture

A molecule mixture or like terms is a mixture containing at least two molecules. The two molecules can be, but not limited to, structurally different (i.e., enantiomers), or compositionally different (e.g., protein isoforms, glycoform, or an antibody with different poly(ethylene glycol) (PEG) modifications), or structurally and compositionally different (e.g., unpurified natural extracts, or unpurified synthetic compounds).

56. Molecule Biosensor Index

A “molecule biosensor index” or like terms is a biosensor index produced by data collected for a molecule. For example, a molecule biosensor index can be made up of a profile of the molecule acting on the panel of cells, and the modulation profile of the molecule against the panels of markers, each panel of markers for a cell in the panel of cells.

57. Molecule DMR Index

A “molecule DMR index” or like terms is a DMR index produced by data collected for a molecule. For example, a molecule biosensor index can be made up of a profile of the molecule acting on the panel of cells, and the modulation profile of the molecule against the panels of markers, each panel of markers for a cell in the panel of cells.

58. Molecule Index

A “molecule index” or like terms is an index related to the molecule.

59. Molecule-Treated Cell

A molecule-treated cell or like terms is a cell that has been exposed to a molecule.

60. Molecule Modulation Index

A “molecule modulation index” or like terms is an index to display the ability of the molecule to modulate the biosensor output signals of the panels of markers acting on the panel of cells. The modulation index is generated by normalizing a specific biosensor output signal parameter of a response of a cell upon stimulation with a marker in the presence of a molecule against that in the absence of any molecule.

61. Molecule Pharmacology

Molecule pharmacology or the like terms refers to the systems cell biology or systems cell pharmacology or mode(s) of action of a molecule acting on a cell. The molecule pharmacology is often characterized by, but not limited, toxicity, ability to influence specific cellular process(es) (e.g., proliferation, differentiation, reactive oxygen species signaling), or ability to modulate a specific cellular target (e.g, PI3K, PKA, PKC, PKG, JAK2, MAPK, MEK2, or actin).

62. Normalizing

Normalizing or like terms means, adjusting data, or a profile, or a response, for example, to remove at least one common variable. For example, if two responses are generated, one for a marker acting a cell and one for a marker and molecule acting on the cell, normalizing would refer to the action of comparing the marker-induced response in the absence of the molecule and the response in the presence of the molecule, and removing the response due to the marker only, such that the normalized response would represent the response due to the modulation of the molecule against the marker. A modulation comparison is produced by normalizing a primary profile of the marker and a secondary profile of the marker in the presence of a molecule (modulation profile).

63. Optional

“Optional” or “optionally” or like terms means that the subsequently described event or circumstance can or cannot occur, and that the description includes instances where the event or circumstance occurs and instances where it does not. For example, the phrase “optionally the composition can comprise a combination” means that the composition may comprise a combination of different molecules or may not include a combination such that the description includes both the combination and the absence of the combination (i.e., individual members of the combination).

64. Or

The word “or” or like terms as used herein means any one member of a particular list and also includes any combination of members of that list.

65. Profile

A profile or like terms refers to the data which is collected for a composition, such as a cell. A profile can be collected from a label free biosensor as described herein.

i. Primary Profile

A “primary profile” or like terms refers to a biosensor response or biosensor output signal or profile which is produced when a molecule contacts a cell. Typically, the primary profile is obtained after normalization of initial cellular response to the net-zero biosensor signal (i.e., baseline)

ii. Secondary Profile

A “secondary profile” or like terms is a biosensor response or biosensor output signal of cells in response to a marker in the presence of a molecule. A secondary profile can be used as an indicator of the ability of the molecule to modulate the marker-induced cellular response or biosensor response.

iii. Modulation Profile

A “modulation profile” or like terms is the comparison between a secondary profile of the marker in the presence of a molecule and the primary profile of the marker in the absence of any molecule. The comparison can be by, for example, subtracting the primary profile from secondary profile or subtracting the secondary profile from the primary profile or normalizing the secondary profile against the primary profile.

66. Panel

A panel or like terms is a predetermined set of specimens (e.g., markers, or cells, or pathways). A panel can be produced from picking specimens from a library.

67. Positive Control

A “positive control” or like terms is a control that shows that the conditions for data collection can lead to data collection.

68. Potentiate

Potentiate, potentiated or like terms refers to an increase of a specific parameter of a biosensor response of a marker in a cell caused by a molecule. By comparing the primary profile of a marker with the secondary profile of the same marker in the same cell in the presence of a molecule, one can calculate the modulation of the marker-induced biosensor response of the cells by the molecule. A positive modulation means the molecule to cause increase in the biosensor signal induced by the marker.

69. Potency

Potency or like terms is a measure of molecule activity expressed in terms of the amount required to produce an effect of given intensity. For example, a highly potent drug evokes a larger response at low concentrations. The potency is proportional to affinity and efficacy. Affinity is the ability of the drug molecule to bind to a receptor.

70. Prodrug

“Prodrug” or the like terms refers to compounds that when metabolized in vivo, undergo conversion to compounds having the desired pharmacological activity. Prodrugs may be prepared by replacing appropriate functionalities present in pharmacologically active compounds with “pro-moieties” as described, for example, in H. Bundgaar, Design of Prodrugs (1985). Examples of prodrugs include ester, ether or amide derivatives of the compounds herein, and their pharmaceutically acceptable salts. For further discussions of prodrugs, see e.g., T. Higuchi and V. Stella “Pro-drugs as Novel Delivery Systems,” ACS Symposium Series 14 (1975) and E, B. Roche ed., Bioreversible Carriers in Drug Design (1987).

71. Publications

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

72. Receptor

A receptor or like terms is a protein molecule embedded in either the plasma membrane or cytoplasm of a cell, to which a mobile signaling (or “signal”) molecule may attach. A molecule which binds to a receptor is called a “ligand,” and may be a peptide (such as a neurotransmitter), a hormone, a pharmaceutical drug, or a toxin, and when such binding occurs, the receptor goes into a conformational change which ordinarily initiates a cellular response. However, some ligands merely block receptors without inducing any response (e.g. antagonists). Ligand-induced changes in receptors result in physiological changes which constitute the biological activity of the ligands.

73. “Robust Biosensor Signal”

A “robust biosensor signal” is a biosensor signal whose amplitude(s) is significantly (such as 3×, 10×, 20×, 100×, or 1000×) above either the noise level, or the negative control response. The negative control response is often the biosensor response of cells after addition of the assay buffer solution (i.e., the vehicle). The noise level is the biosensor signal of cells without further addition of any solution. It is worthy of noting that the cells are always covered with a solution before addition of any solution.

74. “Robust DMR Signal”

A “robust DMR signal” or like terms is a DMR form of a “robust biosensor signal.”

75. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

76. Response

A response or like terms is any reaction to any stimulation.

77. Sample

By sample or like terms is meant an animal, a plant, a fungus, etc.; a natural product, a natural product extract, etc.; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assaYEd as described herein. A sample may also be any body fluid or excretion (for example, but not limited to, blood, urine, stool, saliva, tears, bile) that contains cells or cell components.

78. Salt(s) and Pharmaceutically Acceptable Salt(s)

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of formula I or II with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate. Furthermore, where the compounds of the invention carry an acidic moiety, suitable pharmaceutically acceptable salts thereof may include alkali metal salts, i.e., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (CrC₆) halides (e.g., methyl, ethyl, propyl, and butyl chlorides, bromides, and iodides), dialkyl sulfates (i.e., dimethyl, diethyl, dibuytl, and diamyl sulfates), long chain halides (i.e., decyl, lauryl, myristyl, and stearyl chlorides, bromides, and iodides), arylalkyl halides (i.e., benzyl and phenethyl bromides), and others.

In one embodiment, hemisalts of acids and bases may also be formed, for example, hemisulphate and hemicalcium salts.

The compounds of the invention and their salts may exist in both unsolvated and solvated forms.

79. Signaling Pathway(s)

A “defined pathway” or like terms is a path of a cell from receiving a signal (e.g., an exogenous ligand) to a cellular response (e.g., increased expression of a cellular target). In some cases, receptor activation caused by ligand binding to a receptor is directly coupled to the cell's response to the ligand. For example, the neurotransmitter GABA can activate a cell surface receptor that is part of an ion channel. GABA binding to a GABA A receptor on a neuron opens a chloride-selective ion channel that is part of the receptor. GABA A receptor activation allows negatively charged chloride ions to move into the neuron which inhibits the ability of the neuron to produce action potentials. However, for many cell surface receptors, ligand-receptor interactions are not directly linked to the cell's response. The activated receptor must first interact with other proteins inside the cell before the ultimate physiological effect of the ligand on the cell's behavior is produced. Often, the behavior of a chain of several interacting cell proteins is altered following receptor activation. The entire set of cell changes induced by receptor activation is called a signal transduction mechanism or pathway. The signaling pathway can be either relatively simple or quite complicated.

80. Similarity of Indexes

“Similarity of indexes” or like terms is a term to express the similarity between two indexes, or among at least three indices, one for a molecule, based on the patterns of indices, and/or a matrix of scores. The matrix of scores are strongly related to their counterparts, such as the signatures of the primary profiles of different molecules in corresponding cells, and the nature and percentages of the modulation profiles of different molecules against each marker. For example, higher scores are given to more-similar characters, and lower or negative scores for dissimilar characters. Because there are only three types of modulation, positive, negative and neutral, found in the molecule modulation index, the similarity matrices are relatively simple. For example, a simple matrix will assign identical modulation (e.g., a positive modulation) a score of +1 and non-identical modulation a score of −1.

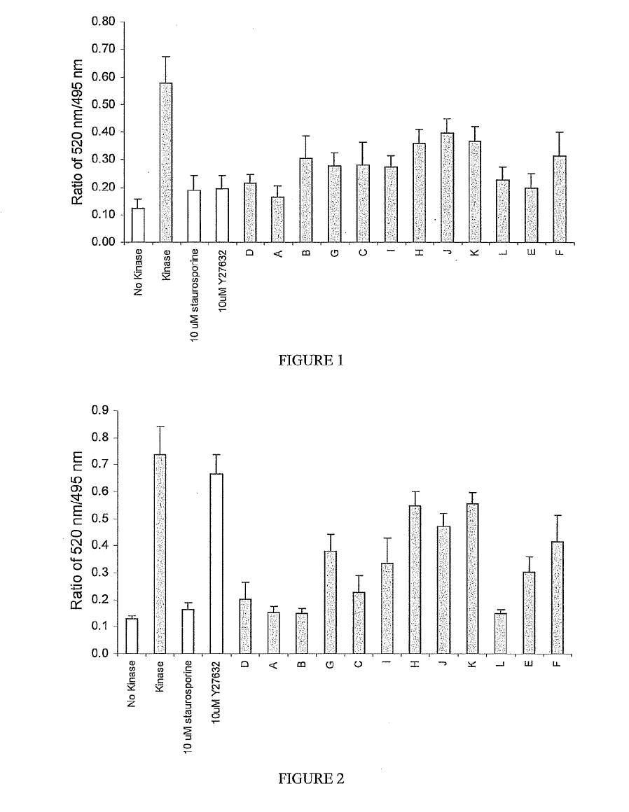

Alternatively, different scores can be given for a type of modulation but with different scales. For example, a positive modulation of 10%, 20%, 30%, 40%, 50%, 60%, 100%, 200%, etc, can be given a score of +1, +2, +3, +4, +5, +6, +10, +20, correspondingly. Conversely, for negative modulation, similar but in opposite score can be given. Following this approach, the modulation index of I against panels of markers, as shown in FIG. 6B, illustrates that the I modulates differently the biosensor response induced by different markers: EGFR in A431 (P-DMR, −60%), EGFR in A431 (N-DMR, −65%), EGF in HT29 (early P-DMR, −70%), EGF in HT29 (late P-DMR, −100%), MTX in HT29 (P-DMR, −60%), and NT in HT29 (P-DMR, −106%). Thus, the score of I modulation index in coordination can be assigned as (˜6, −6.5, −7, −10, −6, −10.6). Similarly, for AG1478 the known EGFR inhibitor, its score in coordination is (−9.2, −10, −10, −10, −6.2, −4.5). By comparing the scores between I and AG1478, one can conclude that both molecules possibly share a similar mode of action in the two cell lines examined, and act as an EGFR inhibitor. Similarly, by comparing the modulation indexes or scores between I and the PDK1/Akt//Flt dual pathway inhibitors, one can conclude that both molecules also possibly share a similar mode of action—a Flt1 inhibitor. Taken together, these similarity analysis suggests that I is a dual EGFR and VEGFR inhibitor, as confirmed by tyrosine kinase inhibition biochemical assays (FIGS. 1, 2 and 3).

81. Solvate

The compounds herein, and the pharmaceutically acceptable salts thereof, may exist in a continuum of solid states ranging from fully amorphous to fully crystalline. They may also exist in unsolvated and solvated forms. The term “solvate” describes a molecular complex comprising the compound and one or more pharmaceutically acceptable solvent molecules (e.g., EtOH). The term “hydrate” is a solvate in which the solvent is water. Pharmaceutically acceptable solvates include those in which the solvent may be isotopically substituted (e.g., D₂O, d₆-acetone, d₆-DMSO).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will be the norm.

The compounds herein, and the pharmaceutically acceptable salts thereof, may also exist as multi-component complexes (other than salts and solvates) in which the compound and at least one other component are present in stoichiometric or non-stoichiomethc amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together. See, e.g., O. Almarsson and M. J. Zaworotko, Chem. Commun., 17:1889-1896 (2004). For a general review of multi-component complexes, see J. K. Haleblian, J. Pharm. Sci. 64(8):1269-88 (1975).

82. Stable

When used with respect to pharmaceutical compositions, the term “stable” or like terms is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

83. Substance

A substance or like terms is any physical object. A material is a substance. Molecules, ligands, markers, cells, proteins, and DNA can be considered substances. A machine or an article would be considered to be made of substances, rather than considered a substance themselves.

84. Subject

As used throughout, by a subject or like terms is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) and mammals, non-human mammals, primates, non-human primates, rodents, birds, reptiles, amphibians, fish, and any other animal. In one aspect, the subject is a mammal such as a primate or a human. The subject can be a non-human.

85. Test Molecule

A test molecule or like terms is a molecule which is used in a method to gain some information about the test molecule. A test molecule can be an unknown or a known molecule.

86. Treating

Treating or treatment or like terms can be used in at least two ways. First, treating or treatment or like terms can refer to administration or action taken towards a subject. Second, treating or treatment or like terms can refer to mixing any two things together, such as any two or more substances together, such as a molecule and a cell. This mixing will bring the at least two substances together such that a contact between them can take place.

When treating or treatment or like terms is used in the context of a subject with a disease, it does not imply a cure or even a reduction of a symptom for example. When the term therapeutic or like terms is used in conjunction with treating or treatment or like terms, it means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

87. Trigger

A trigger or like terms refers to the act of setting off or initiating an event, such as a response.

88. Values

Specific and preferred values disclosed for components, ingredients, additives, cell types, markers, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

Thus, the disclosed methods, compositions, articles, and machines, can be combined in a manner to comprise, consist of, or consist essentially of, the various components, steps, molecules, and composition, and the like, discussed herein. They can be used, for example, in methods for characterizing a molecule including a ligand as defined herein; a method of producing an index as defined herein; or a method of drug discovery as defined herein.

89. Unknown Molecule

An unknown molecule or like terms is a molecule with unknown biological/pharmacological/physiological/pathophysiological activity, but with known or unknown chemical structure.

90. Optimizing

Optimizing refers to a process of making better or checking to see if it something or some process can be made better.

91. Therapeutic Efficacy

Therapeutic efficacy refers to the degree or extent of results from a treatment of a subject.

92. Disease Marker

A disease marker is any reagent, molecule, substance etc, that can be used for identifying, diagnosing, or prognosing is for the EGFR or VEGFR related disease.

93. Toxicity Marker

A toxicity marker is any reagent, molecule, substance etc. that can be used for identifying, diagnosing, prognosing a level of toxicity of a substance, in, for example, an organism or cell or tissue or organ.

94. Analytical Methods

An analytical method is, for example, a method which measures a molecule or substance. For example, gas chromatography, gel permeation chromatography, high resolution gas chromoatography, high resolution mass spectrometry, or mass spectrometry is analytical methods.

95. Toxicity

Toxicity is the degree to which a substance, molecule, is able to damage something, such as a cell, a tissue, an organ, or a whole organism, that has been exposed to the substance or molecule. For example, the liver, or cells in the liver, hepatocytes, can be damaged by certain substances.

### I. Examples

### 1. Example 1

### Chemical Synthesis and Characterization

### i. Synthesis of Compound A, B, C, D, E, and F

These compounds were synthesized using similar protocol as shown in Scheme 1. The detail synthesis procedure of compound A was used as an example. All compounds were purified using High Performance Liquid Chromatograph (HPLC) and characterized using both ¹H NMR and ¹³C NMR.

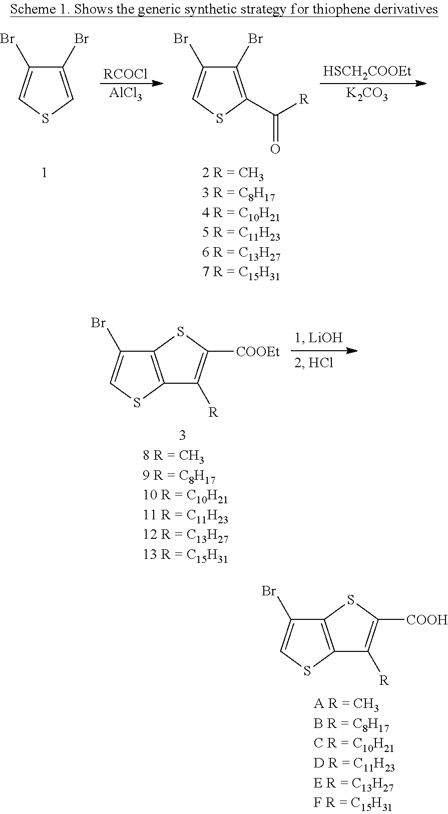

### (a) Synthesis of 3,4-Dibromothienyl-2-methyl ketone (2)

To a mixture of 3,4-dibromothiophene 1 (72.60 g, 0.30 mol) and AlCl₃ (92.46 g, 0.69 mol) in CH₂Cl₂ (300 mL) at 0° C., acetyl chloride (24.73 g, 0.32 mol) was added dropwise under a nitrogen stream. This was stirred for 2 to 3 hours until no starting materials could be detected by GC/MS. The mixture was then poured into HCl (500 mL, 6M) and the organic was extracted with CH₂Cl₂ (2×300 mL). The combined organic solution was washed with brine (2×150 mL) and water (150 mL). After drying over anhydrous MgSO₄, the solvent was evaporated. A low melting point solid was collected and was pure enough to be used without further purification (80.80 g, 95%). mp 75-78° C. ¹H NMR (300 MHz, CD₂Cl₂) δ 7.67 (s, 1H), 2.69 (s, 3H). ¹³C NMR (300 MHz, CD₂Cl₂) 189.6, 140.6, 130.2, 118.0, 117.3, 29.7, HRMS (ESI) m/z calcd for [C₆H₄Br₂OS] 281.8300, observed; 282.9000.

### (b) Synthesis of 3-Methyl-6-bromo-ethylthieno[3,2-b]thiophene-2-carboxylate (8)

Compound 2 (80.80 g, 0.29 mol) was mixed with K₂CO₃ (196.70 g, 1.43 mol) and DMF (250 mL) in a three neck flask equipped with a condenser and addition funnel. To this mixture ethyl mercaptoacetate (32.80 mL, 0.30 mol) was added dropwise at 60-70° C. A catalytic amount of 18-crown-6 (20 mg) was used as catalyst. The mixture was heated at 60-70° C. overnight until no starting materials were detected by GC/MS. The mixture then was poured into water (1000 mL) and a light YEllow solid was formed. After filtration, the solid washed with water (3×500 mL) and filtrated. The collected solid was washed with methanol (300 mL) and found to be pure enough for the next reaction (78.40 g, 90%). mp 91-92° C. ¹H NMR (300 MHz, CD₂Cl₂) δ 7.48 (s, 1H), 4.36 (q, 2H), 2.63 (s, 3H), 1.38 (t, 3H). ¹³C NMR (300 MHz, CD₂Cl₂) 159.4, 137.9, 137.6, 135.1, 125.7, 124.2, 99.7, 57.8, 11.1, 10.7. HRMS (ESI) m/z calcd for [C₁₀H₉BrO₂S₂] 303.9200, found 303.3000

### (c) Synthesis of 3-Methyl-thieno[3,2-b]thiophene-2-carboxylic acid (A)

Compound 8 (78.40 g, 0.26 mol) was dissolved into a mixture of THF (400 mL), methanol (50 mL) and LiOH (100 mL, 10% solution). This mixture was refluxed overnight and poured into concentrated hydrochloric acid (300 mL). The acid mixture was then diluted to 1000 mL with water. Solid was filtrated and washed with water (3×500 mL). The light YEllow solid was washed with methanol (300 mL) and dried under vacuum overnight (68.10 g. 96%). mp 280-282° C. ¹H NMR (300 MHz, DMSO) δ 8.08 (s, 1H), 2.60 (s, 3H). ¹³C NMR (300 MHz, DMSO) 163.6, 140.7, 140.1, 137.7, 129.7, 129.2, 102.1, 14.2. HRMS (MALDI) m/z calcd for [C₈H₅BrO₂S₂—H₂O+H] 258.8887, found 258.8883.

### ii. Synthesis of Compound J

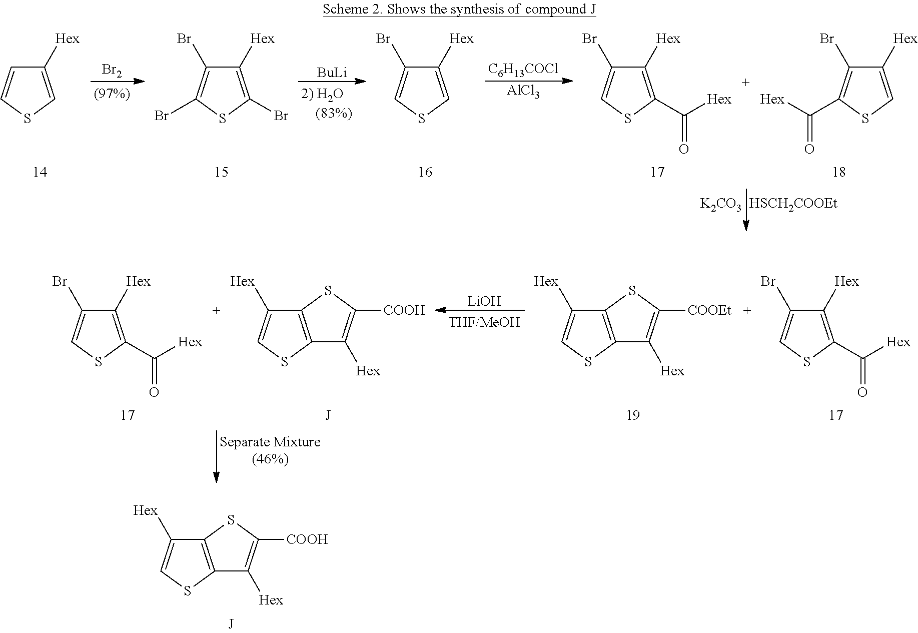

J was synthesized using the protocol as shown in Scheme 2.

### (a) Synthesis of 2,4,5-Tribromo-3-hexylthiophene (15)

3-hexylthiophene 14 (100.00 g, 0.60 mol) was mixed with 200 mL acetic acid. To this mixture, bromine (88.00 mL, 1.33 mol) was added dropwise. After finishing the addition of bromine, the mixture was then stirred at room temperature for 4 hours and heated to 60-70° C. overnight. The final mixture was poured into 800 mL ice water and neutralized with NaOH solution (6M). The organic was extracted with ethyl acetate (3×100 mL). The combined organic was washed with brine (2×100 mL), water (100 mL) and dried over anhydrous MgSO₄. After evaporating solvent, 234.00 g (97%) of crude product was obtained. This product was found pure enough for the next reaction. GC/MS: 404 (M−1). ¹H NMR (300 MHz, CD₂Cl₂) δ 2.64 (t, 2H), 1.51 (m, 2H), 1.32 (m, 6H), 0.89 (t, 3H). ¹³C NMR (300 MHz, CD₂Cl₂ 143.7, 117.9, 111.5, 110.2, 33.6, 32.9, 31.00, 30.5, 24.7, 16.0.

### (b) Synthesis of 3-Bromo-4-hexylthiophene (16)

Compound 15 (70.00 g, 0.17 mol) was mixed with dry THF (400 mL). To this mixture n-butyllithium (138 mL, 2.5M in hexane, 0.35 mol) was added dropwise at −78° C. under argon. After finishing the addition, the mixture was stirred another 10 minutes and water was added to quench the reaction. The THF was evaporated and organic was extracted with ethyl acetate (2×100 mL). The combined organic layer was washed by brine (2×100 mL), water (70 mL) and dried over anhydrous MgSO₄. After evaporating solvent, the crude product was purified by vacuum distillation at 72-74° C./0.17 millibar giving 35.30 g (83%). GC/MS: 246 (M−1). ¹H NMR (300 MHz, CD₂Cl₂) δ 7.22 (s, 1H), 6.96 (s, 1H), 2.57 (t, 2H), 1.61 (m, 2H), 1.32 (m, 6H), 0.88 (t, 3H). ¹³C NMR (300 MHz, CD₂Cl₂ 141.9, 122.9, 121.0, 112.9, 31.9, 30.1, 29.5, 29.2, 22.9, 14.1.

### (c) Synthesis of 1-(4-Bromo-3-hexyl-2-thienyl)heptanone (17) and 1-(3-Bromo-4-hexyl-2-thienyl)heptanone (18)

To a mixture of compound 16 (24.70 g, 0.10 mol) and AlCl₃ (26.80 g, 0.20 mol) in dry CH₂Cl₂ (100 mL), heptanoyl chloride (14.90 g, 0.10 mol) was added dropwise at room temperature. This mixture was stirred for two hours and GC/MS shown a 1:3 mixtures of target compound 18 and heptanone 17 were formed. This mixture was poured into HCl (6M) and washed with water (3×50 mL). The organic mixture then was dried over anhydrous MgSO₄. After evaporating solvent, 34.70 g of 17 and 18 mixtures of crude products was obtained as confirmed by GC/MS and used for the next reaction without separation.

### (d) Synthesis of 3,6-Dihexyl-thieno[3,2-b]thiophene-2-carboxylic acid (J)

A mixtures of compounds 17 and 18 (66.50 g, 0.19 mol) was mixed with K₂CO₃ (53.60 g, 0.39 mol) and a catalytic amount of 18-Crown-6 in 200 mL DMF. To this mixture, ethyl mercaptoacetate (20.30 mL, 0.19 mol) was added dropwise at 60-70° C. The mixture was stirred at this temperature overnight and poured into water (800 mL). The organic was extracted with ethyl acetate (3×100 mL), washed with brine (2×100 mL) and water (100 mL). The organic layer was collected and solvent was evaporated. The residue included 3,6-dihexyl-thieno[3,2-b]thiophene-2-carboxylic acetate 19 and compound 17 as confirmed by GC/MS. This mixture was then dissolved in THF (300 mL). To this THF solution LiOH (84 mL, 10% w/w solution in water), MeOH (50 mL) and a catalytic amount of tetrabutylammonium iodide were added. The mixture was refluxed for 3 hours and the solvent then was evaporated. The residue was then acidified with concentrated HCl (50 mL). The organic was extracted with ethyl acetate (3×100 mL) after dilution by water. The combined organic layer was washed with brine (2×100 mL), water (100 mL) and dried over anhydrous MgSO₄. After evaporating solvent, the pure compound J was obtained by silica gel column chromatography (5% ethyl acetate in hexane and then 20% ethyl acetate in hexane to elute). Yield 30.00 grams (46%) (Calculated from mixture). ¹H NMR (300 MHz, CD₂Cl₂) δ 7.24 (s, 1H), 3.18 (t, 2H), 2.73 (t, 2H), 1.75 (m, 4H), 1.34 (m, 14H), 0.89 (m, 6H). ¹³C NMR (300 MHz, CD₂Cl₂ 169.2, 146.3, 143.1, 141.5, 136.1, 126.7, 126.1, 32.0, 29.7 (6C), 23.0, 14.2. HRMS (MALDI) m/z calcd for [C₁₉H₂₈O₂S₂—H₂O+H] 335.1503, found 335.1508.

### iii. Synthesis of Compound K

Compound K was synthesized using the protocol as showed in Scheme 3.

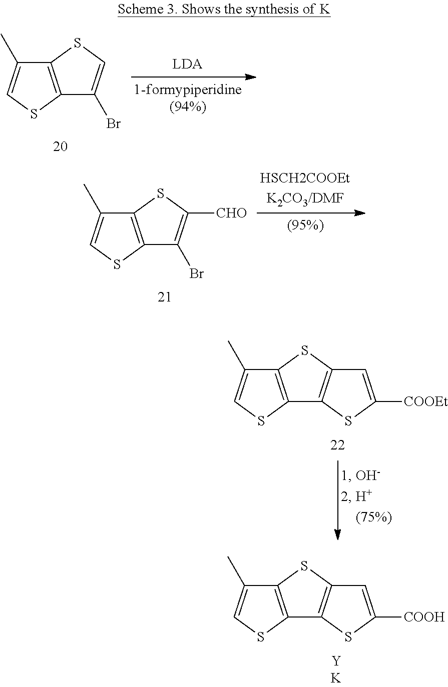

### (a) Synthesis of 2-Formyl,3-bromo-6-methylthieno[3,2-b]thiophene (21)

To a mixture of diisopropylamine (22.24 g, 0.22 mol) in dry THF (200 mL), n-butyllithium (80.00 mL, 2.50M in hexane, 0.20 mol) was added dropwise at 0° C. under argon. The freshly made lithium diisopropylamide (LDA) was stirred for 30 minutes at 0° C., and compound 20 (42.40 g, 0.18 mol) was dissolved in dry THF (100 mL) and added dropwise. The resulting mixture was stirred one hour before 1-formylpiperidine (23.70 g, 0.21 mol) was added dropwise. The final mixture was stirred overnight at room temperature before being poured into HCl (200 mL, 10% solution). The solid formed was filtered and washed with water (3×500 mL). The crude solid product was then washed with ethanol (300 mL) and dried under vacuum to give 44.50 grams. (94%) mp 127-129° C. ¹H NMR (300 MHz, CD₂Cl₂) δ 10.01 (s, 1H), 7.37 (s, 1H), 2.37 (s, 3H). ¹³C NMR (300 MHz, CD₂Cl₂) 183.7, 145.2, 13, 141.4, 137.7, 131.8, 129.7, 113.9, 14.7. HRMS (ESI) m/z calcd for [C₈H₅BrOS₂] 259.9000, found 260.1000.

### (b) Synthesis of 2-Carboethoxyl-5-methyldithieno[3,2-b:2′3′-d]thiophene (22)

Compound 21 (44.50 g, 0.17 mol), K₂CO₃ (113.00 g, 0.82 mol) and ethyl mercaptoacetate (22.90 g, 0.19 mol) were reacted in DMF (300 mL). Compound 22 was obtained as a yellow solid 45.60 g (95%). mp 86-88° C. ¹H NMR (300 MHz, CD₂Cl₂) δ 7.99 (s, 1H), 7.13 (s, 1H), 4.38 (q, 2H), 2.38 (s, 3H), 1.39 (t, 3H). ¹³C NMR (300 MHz, CD₂Cl₂) 162.8, 146.5, 141.0, 136.9, 134.0, 131.8, 130.4, 127.5, 124.3, 62.0, 14.9. HRMS (MALDI) m/z calcd for [C₁₂H₁₀O₂S₃] 281.9843, found 281.9838.

### (c) Synthesis of 5-methyldithieno[3,2-b:2′3′-d] thiophene-2-carboxyl acid (K)

Compound 22 (45.60 g, 0.16 mol), LiOH (10% in water, 60 mL), THF (300 mL) and methanol (50 mL) were refluxed overnight. After purification, the light yellow powder K was obtained (31.00 g, 75%). mp 289-290° C. ¹H NMR (300 MHz, DMSO) δ 8.19 (s, 1H), 7.49 (s, 1H), 2.55 (s, 3H). HRMS (MALDI) m/z calcd for [C₁₀H₆O₂S₃] 254.9608, found 254.9604.

### iii. Synthesis of Compound H

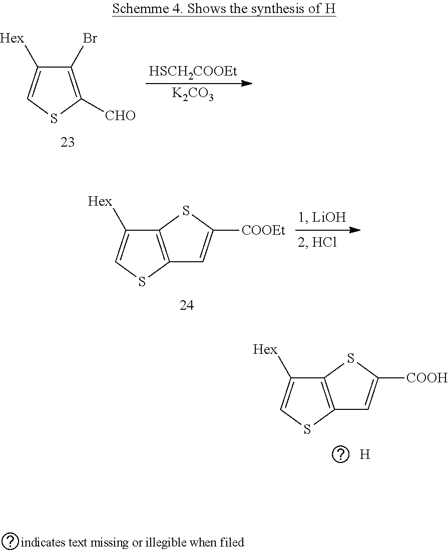

### (a) Synthesis of 6-hexyl-ethylthieno[3,2-b]thiophene-2-carboxylate (24)

Compound 23 (105.2 g, 0.383 mol) was mixed with K₂CO₃ (84.60 g, 0.61 mol) and DMF (300 mL) in a three neck flask equipped with a condenser and an addition funnel. To this mixture, ethyl mercaptoacetate (16.42 mL, 0.15 mol) was added dropwise at room temperature. The mixture was stirred at room temperature overnight until no starting materials were detected by GC/MS. The mixture was then poured into water (500 mL) and extracted by ethyl acetate (2×200 ml). Organic extracts were washed by brine (3×300 ml), and dried over MgSO₄. After evaporating the solvent, the brownish crude target was obtained and found to be pure enough for the next reaction (113.4 g, 100%). GC/MS 297[M+].

### (b) Synthesis of 6-hexyl-thieno[3,2-b]thiophene-2-carboxylic acid (H)

Compound 24 (113.4 g, 0.383 mol) was dissolved into a mixture of THF (300 mL) and LiOH (200 mL, 10% solution). This mixture was refluxed overnight and poured into concentrated hydrochloric acid (300 mL). The acid mixture was then diluted to 1000 mL with water. Solid was filtrated and washed with water (3×500 mL). The light yellow solid of H was recrystallized from hexane and dried under vacuum overnight (48.7 g. 69.5%). GC-MS 224 [M−COOH].

### iv. Synthesis of Compound L

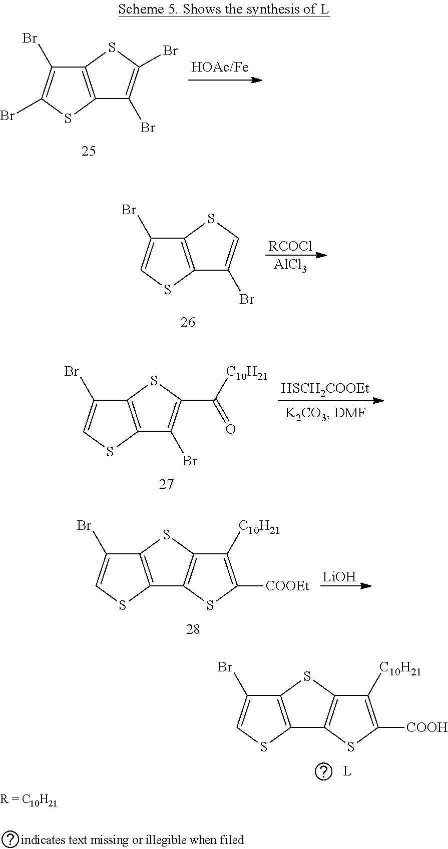

### (a) Synthesis of Compound 26

To a refluxing solution of compound 25 (79.80 g, 0.175 mol) in a mixed solvents of HOAC (400 mL), water (10 mL), and concentrated HCl (10 mL) in a 1 L two neck round-bottomed flask, 11.44 g (0.175 mol) of Zn powder was added. After all the bubbles were gone, additional 11.44 g (0.175 mol) of Zn powder was added. More portions of Zn powder in such an mount were added until no bubbles appeared after the addition of Zn powder and a clear solution was formed. A hot filtration was then carried out. After the filtrate was cooled to room temperature, a white precipitate formed was collected. This white precipitate was washed by water to yield 44.00 g (85%) of compound 26 after being vacuum-dried. GC/MS 297[M+].

### (b) Synthesis of Compound 27

To a mixture of compound 26 (12.43 g, 0.0417 mol) and AlCl₃ (12.78 g, 0.0959 mol) in CH₂Cl₂ (70 mL) at 0° C., n-Undecanoyl chloride (10.25 g, 0.0500 mol) was added dropwise under a nitrogen stream. This was stirred at 0° C. for about 12 hours until only small amount of starting materials could be detected by GC/MS. The mixture was then poured into HCl (200 mL, 6M) and the organic was extracted with hexanes/CH₂Cl₂ (1:1) (2×200 mL). The combined organic solution was washed with brine (2×100 mL) and water (100 mL). After drying over anhydrous MgSO₄, the solvent was evaporated. A low melting point solid was collected and was recrystallized from EtOH to yield 18.32 g, 94%). GC-MS 465[M+].

### (c) Synthesis of Compound 28

Compound 27 (14.05 g, 0.0301 mol), K₂CO₃ (16.65 g, 0.121 mol) added into DMF (100 mL) in a three neck flask equipped with a condenser and addition funnel. To this mixture ethyl mercaptoacetate (3.80 mL, 0.03166 mol) was added dropwise at 60-70° C. A catalytic amount of 18-crown-6 (20 mg) was used as catalyst. The mixture was heated at 60-70° C. overnight until no starting materials were detected by GC/MS. The mixture then was poured into water (300 mL) and extracted by ethyl acetate (2×100 ml). Organic extracts were washed by brine (3×400 ml), and dried by MgSO₄. After evaporating the solvent, the brownish crude target was obtained and found to be pure enough for the next reaction (14.93 g, 100%). GC/MS 486[M+].

### (d) Synthesis of L

Compound 28 (14.68 g, 0.0301 mol), LiOH (10% in water, 22 mL), THF (100 mL) and methanol (10 mL) were refluxed overnight. This mixture was then cooled to room temperature and poured into concentrated hydrochloric acid (100 mL). The acid mixture was then diluted to 200 mL with water. Solid was filtrated and washed with water (3×200 mL). The light yellow solid of L was washed with methanol (100 mL) and dried under vacuum overnight (13.28 g. 96%). GC-MS 416 [M-COOH].

### 2. Example 2

### Label-Free Optical Biosensor Cellular Assay Characterization of Compounds

EGF is the natural agonist for EGF receptors which is endogenously expressed in both A431 and HT29 cells. Thus, both cell lines were chosen to study the ability of compounds to inhibit EGF-induced signaling. A431 cells primarily endogenously expresses EGFR (erbB1), while HT29 cells predominately endogenously expresses her2/neu, and also expresses erbB1.

The NTS1 natural agonist neurotensin is known to activate the endogenous NTS1/NTS3 heterodimers in HT29 cells. Although it is unknown in literature whether the activation of NTS1 receptors transactivates EGF receptor or not, our study using Epic® cellular assays indicated that stimulation of HT29 with NT led to the activation of NTS1 receptors, and subsequently transactivated EGF receptors (data not shown). Thus, neurotensin was chosen as an indirect marker for identifying potential EGFR inhibitors.

HT29 expresses VEGFR1 (Flt1 receptor), but not other VEGF receptors. However, we have found that the Flt1 natural agonist VEGF up to 100 nM only triggered a very small DMR signal in un-starved HT29 cells (data not shown). Since in acute myeloid leukemia cells, the hERG ion channel is postulated to form a super signaling complex with FLT1 receptor (S. Pillozzi, et al. VEGFR-1 (Flt-1), 131 integrin, and hERK K+ channel for a macromolecular signaling complex in acute myeloid leukemia: role in cell migration and clinical outcome. Blood 2007, 15, 1238-1250), and HT29 endogenously expresses both Flt1 and hERG1 ion channels, thus the activation of hERG ion channel in HT29 by the known hERG activator mallotoxin can be used an indirect readout for screening potential VEGFR inhibitors.

Optical biosensors primarily employ a surface-bound electromagnetic wave to characterize cellular responses. The surface-bound waves can be achieved on metallic substrates using either light excited surface plasmons (surface plasmon resonance, SPR) or on dielectric substrates using diffraction grating coupled waveguide mode resonances (resonance waveguide grating, RWG). For SPR including mid-IR SPR, the readout is the resonance angle at which a minimal in intensity of reflected light occurs. Similarly, for RWG biosensor including photonic crystal biosensor, the readout is the resonance angle or wavelength at which a maximum incoupling efficiency is achieved. The resonance angle or wavelength is a function of the local refractive index at or near the sensor surface. Unlike SPR, which is limited to a few of flow channels for assaying, RWG biosensors are amenable for high throughput screening (HTS) and cellular assays, due to recent advancements in instrumentation and assays. In a typical RWG, the cells are directly placed into a well of a microtiter plate in which a biosensor consisting of a material with high refractive index is embedded. Local changes in the refractive index lead to a dynamic mass redistribution (DMR) signal of live cells upon stimulation. These biosensors have been used to study diverse cellular processes including receptor biology, ligand pharmacology, and cell adhesion.

Using the RWG biosensor Corning® Epic® system, all compounds were systematically characterized for their ability to modulate EGFR and VEGFR activity in live cells. The Epic® system consists of a temperature-control unit, an optical detection unit, with an on-board liquid handling unit with robotics, or an external liquid accessory system with robotics. The detection unit is centered on integrated fiber optics, and enables kinetic measures of cellular responses with a time interval of ˜7 or 15 sec. The compound solutions were introduced by using either the on-board liquid handling unit, or the external liquid accessory system; both of which use conventional pippetting system.

i. Materials and Methods

a. Materials

Mallotoxin was obtained from BioMol International Inc (Plymouth Meeting, Pa.). Epidermal growth factor (EGF), and neurotensin was obtained from BaChem Americas Inc. (Torrance, Calif.). Lavendustin A and A1478 was obtained from BioMol. VEGF Receptor 2 Kinase Inhibitor I (Z)-3-[(2,4-Dimethyl-3-(ethoxycarbonyl)pyrrol-5-yl)methylidenyl]indolin-2-one, VEGF Receptor 2 Kinase Inhibitor II (Z)-5-Bromo-3-(4,5,6,7-tetrahydro-1H-indol-2-ylmethylene)-1,3-dihydroindol-2-one, Flt-3 Inhibitor III (5-Phenyl-thiazol-2-yl)-(4-(2-pyrrolidin-1-yl-ethoxy)-phenyl)-amine, and PDK1/Akt/Flt Dual Pathway Inhibitor 6H-Indeno[1,2-e]tetrazolo[1,5-b][1,2,4]triazin-6-one & 10H-Indeno[2,1-e]tetrazolo[1,5-b][1,2,4]triazin-10-one were obtained from EMD Biosciences (Gibbstown, N.J.). Epic® 384 biosensor microplates cell culture compatible were obtained from Corning Inc. (Corning, N.Y.).

b. Cell Culture

All cell lines were obtained from American Type Cell Culture (Manassas, Va.). The cell culture medium was as follows: (1) Dulbecco's modified Eagle's medium (DMEM) supplemented with 10% fetal bovine serum (FBS), 4.5 g/liter glucose, 2 mM glutamine, and antibiotics for human epidermoid carcinoma A431; and (2) McCoy's 5a Medium Modified supplemented with 10% FBS, 4.5 g/liter glucose, 2 mM glutamine, and antibiotics for human colorectal adenocarcinoma HT29.

Cells were typically grown using ˜1 to 2×10⁴ cells per well at passage 3 to 15 suspended in 50 μl of the corresponding culture medium in the biosensor microplate, and were cultured at 37° C. under air/5% CO₂ for ˜1 day. Except for A431 which underwent one day culture followed by one starvation in serum free medium, all other cells were directly assayed without starvation. The confluency for all cells at the time of assays was ˜95% to 100%.

c. Optical Biosensor System and Cell Assays

Epic® beta version wavelength interrogation system (Corning Inc., Corning, N.Y.) was used for whole cell sensing. This system consists of a temperature-control unit, an optical detection unit, and an on-board liquid handling unit with robotics. The detection unit is centered on integrated fiber optics, and enables kinetic measures of cellular responses with a time interval of ˜15 sec.

The RWG biosensor is capable of detecting minute changes in local index of refraction near the sensor surface. Since the local index of refraction within a cell is a function of density and its distribution of biomass (e.g., proteins, molecular complexes), the biosensor exploits its evanescent wave to non-invasively detect ligand-induced dynamic mass redistribution in native cells. The evanescent wave extends into the cells and exponentially decays over distance, leading to a characteristic sensing volume of ˜150 nm, implying that any optical response mediated through the receptor activation only represents an average over the portion of the cell that the evanescent wave is sampling. The aggregation of many cellular events downstream the receptor activation determines the kinetics and amplitudes of a ligand-induced DMR.

For biosensor cellular assays, molecule solutions were made by diluting the stored concentrated solutions with the HBSS (1× Hanks balanced salt solution, plus 20 mM Hepes, pH 7.1), and transferred into a 384well polypropylene molecule storage plate to prepare a molecule source plate. Both molecule and marker source plates were made separately when a two-step assay was performed. In parallel, the cells were washed twice with the HBSS and maintained in 30 μl of the HBSS to prepare a cell assay plate. Both the cell assay plate and the molecule and marker source plate(s) were then incubated in the hotel of the reader system. After ˜1 hr of incubation the baseline wavelengths of all biosensors in the cell assay microplate were recorded and normalized to zero. Afterwards, a 2 to 10 minute continuous recording was carried out to establish a baseline, and to ensure that the cells reached a steady state. Cellular responses were then triggered by pipetting 10 μl of the marker solutions into the cell assay plate using the on-board liquid handler.

To study the influence of molecules on a marker-induced response, a second stimulation with the marker at a fixed dose (typically at EC80 or EC100) was applied. The resonant wavelengths of all biosensors in the microplate were normalized again to establish a second baseline, right before the second stimulation. The two stimulations were usually separated by ˜1 hr.

All studies were carried out at a controlled temperature (28° C.). At least two independent sets of experiments, each with at least three replicates, were performed. The assay coefficient of variation was found to be <10%.

d. Time Resolved FRET (Fluorescence Resonance Energy Transfer) Assays

LanthaScreen TR-FRET kinase assay reagents and the recombinant EGFR, VEGFR1 (FLT1), VEGFR2 (KDR) were purchased from Invitrogen. Known kinase inhibitors were used as positive controls. Staurosporine was from Sigma, EGFR inhibitor Iressa was from Tocris. Compound library was diluted with kinase reaction buffer with final concentration of 25 μM with 1% DMSO.

Kinase reaction was performed in Corning 3676 low volume 384 well black round bottom assay plate in 10 μl/well. Each EGFR reaction contains 51 ng/ml kinase, 2.4 μM ATP and 0.2 μM substrate. Each VEGFR1 (FLT1) reaction contains 46.8 ng/ml kinase, 66.7 μM ATP and 0.2 μM substrate. Each VEGFR2 (KDR) reaction contains 4 ng/ml kinase, 13 μM ATP and 0.2 μM substrate. Kinase reactions are allowed to proceed for 1 hour at room temperature before a 10 μl preparation of EDTA (20 mM) and Tb-labeled antibody (4 nM) in TR-FRET dilution buffer are added. The final concentration of antibody in the assay well is 2 nM, and the final concentration of EDTA is 10 mM. The plate is allowed to incubate at room temperature for at least 30 minutes before being read on a Tecan SafireII plate reader configured for LanthaScreen™ TR-FRET (Excitation at 340 nm, emission at 495 nm and 520 nm). The final readout is the emission ratio 520 nm/495 nm. Each data point is the average of 4 replicates.

e. Cell Proliferation Assays

Proliferation was measured using the CellTiter-Glo Luminescent Cell Viability Assay (Technical Bulletin #288, Promega, Madison, Wis.). When added to cells, the assay reagent produces luminescence in the presence of ATP from viable cells. Cells were plated in 96-well Corning Costar TCT (tissue culture treated) plates at a density of 10,000 cells/well and incubated for 24 h. Test samples were dissolved in dimethyl sulfoxide (DMSO) by sonication, filter sterilized and diluted with media to the desired treatment concentration. Cells were treated with 100 μl control media, or test samples, and incubated for 48 h drug exposure duration. At the end of 48 h, plates were equilibrated at room temperature for 30 min, 100 μl of the assay reagent was added to each well and cell lysis was induced on an orbital shaker for 2 min. Plates were incubated at room temperature for 10 min to stabilize the luminescence signal and results were read on an Perkin Elmer Vector 3 Microplate Reader. All plates had control wells containing medium without cells to obtain a value for background luminescence. Data are expressed as for three replications.

ii. Results

The DMR modulation index were generated against 4 markers across two distinct cell lines—the EGFR agonist EGF in A431 (EGF at 32 nM), and the EGFR agonist EGF in HT29 (EGF at 2 nM), the hERG activator mallotoxin (MTX) in HT29 (MTX at 16 micromlar), and the NTS1/NTS3 agonist neurotensin (NT) in HT29 (NT at 2 nM). The EGF responses in A431 cells include the early P-DMR event (˜5 min after EGF stimulation) and the subsequent N-DMR event (˜30 min after EGF stimulation). The EGF responses in HT29 include the early P-DMR event (˜5 min after EGF stimulation) and the late P-DMR event (˜50 min after EGF stimulation), whereas the mallotoxin response in HT29 is the P-DMR response 50 min after MTX stimulation, and the NT response in HT29 is the P-DMR 50 min after NT stimulation. In all cases, the amplitudes of respective DMR events were used as the basis to calculate the percentages of modulation by each inhibitor.

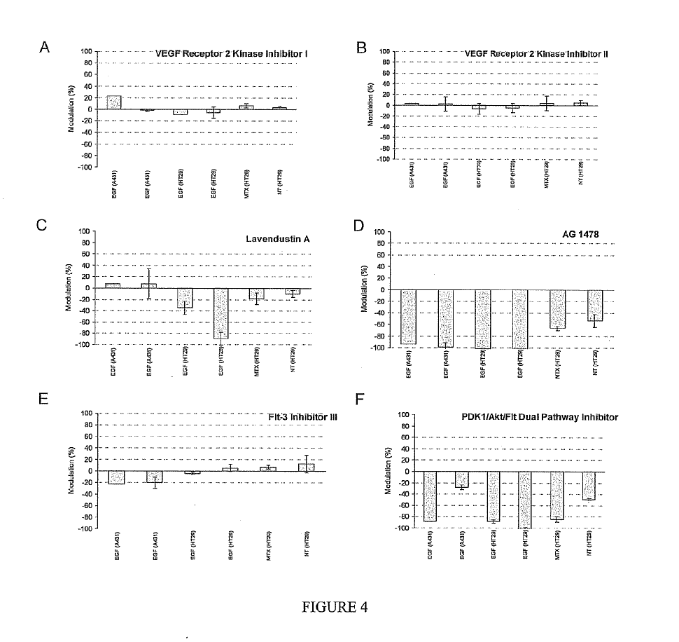

As shown in FIGS. 4A and B, the two known VEGFR2 tyrosine kinase inhibitors (I and II) had little impact on the DMR signals of all markers examined. This is expected since HT29 only expresses VEGFR1 receptors, and there is no any literature reports showing that the EGFR, hERG ion channels, or NTS1 receptors directly cross-talk with VEGFR2.

As shown in FIGS. 4C and D, the two known EGFR tyrosine kinase inhibitors (lavendustin A and A1478) exhibited different modulation patterns. Lavendustin A is a weak EGFR inhibitor, whereas A1478 is a potent EGFR inhibitor. The partial inhibition of the NT response in HT29 by AG1478 suggests that the activation of NTS1 indeed transactivates EGFR. The partial inhibition of the MTX responses in HT29 by A1478 is also expected since the complete inhibition of EGFR basal activity can attenuate the hERG activity.

As shown in FIG. 4E, the known Flt3 inhibitor III also had little impact on either DMR signal. However, the dual PDK1/AKT/Flt1 pathway inhibitor almost completely attenuated the early P-DMR event in the EGF response in A431, and both the early and late P-DMR events of the EGF response in HT29. This is expected since PDK1/Akt pathway is downstream of EGFR signaling. However, the dual pathway inhibitor also almost completely attenuated the MTX response in HT29, suggesting that it indeed also acts as a VEGFR1 inhibitor. The possible reason behind is that VEGFR1/Flt1 is also complexed with hERG in HT29 cells, similar to those formed in leukemia cancer cells. The activation of hERG by MTX may also transactivate VEGFR1.

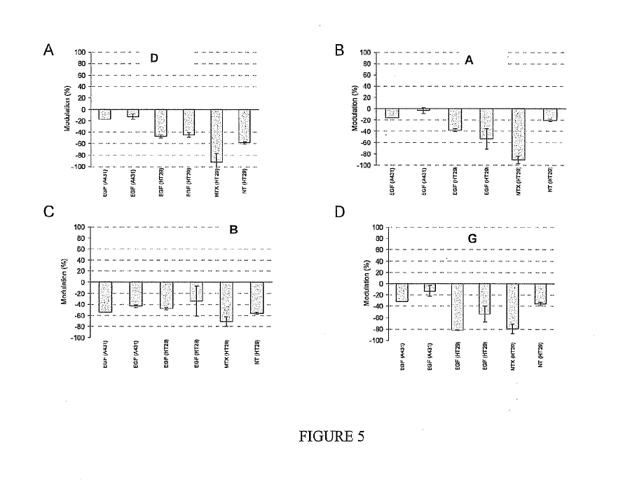

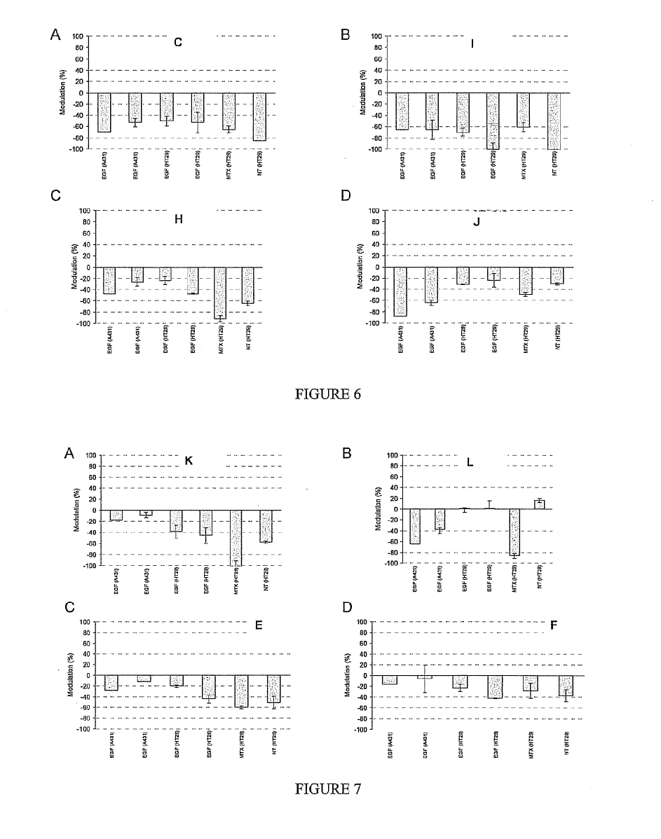

Based on these modulation indices of distinct classes of EGFR and VEGFR inhibitors, dual EGFR and VEGFR inhibitors should also significantly attenuated the DMR signals of all 4 markers for the two cell lines A431 and HT29. Approximately 600 internally synthesized compounds were screened using the 4 marker panel. Based on the similarity in DMR modulation index, about 45 potential dual EGFR and VEGFR inhibitors were identified. Among these hits, structure-activity relationship analysis further identified a family of thieno[3,2-β]-thiophene compounds, 12 in total, that share similar cellular pharmacology using the label-free biosensor cellular assays. These compounds' DMR modulation indexes were presented in FIGS. 5-7. Results showed that these 12 compounds gave rise to similar DMR index against the 4 marker panel. The variation in modulation percentage for a specific marker DMR signal is due to the potency of these chemicals, as well as the phenotypic pharmacology and polypharmacology of these compounds.

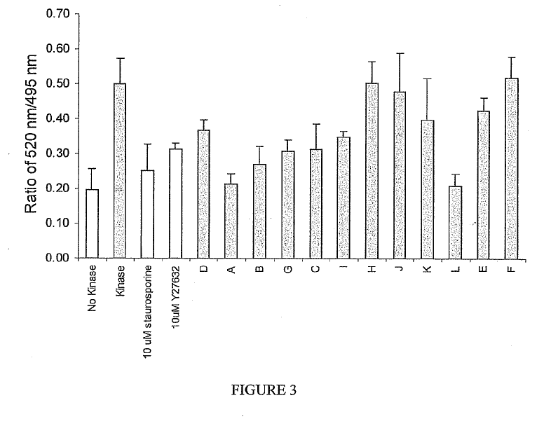

Kinase biochemical assays were used to confirm these chemicals as dual EGFR and VEGFR inhibitors. LanthaScreen TR-FRET kinase assay reagents and the recombinant EGFR, VEGFR1 (FLT1), VEGFR2 (KDR) were used. The assay protocols recommended by the supplier were used after optimization. The main results were summarized in FIGS. 1-3. Known kinase inhibitors were used as positive controls. Results showed that the pan kinase inhibitor staurosporine at 10 micromolar completely inhibited the FRET signal of each kinase tested. The potent and selective Iressa at 100 nM also completely inhibited the FRET signal for EGFR tyrosine kinase (FIG. 1), but not VEGFR1 tyrosine kinase (FIG. 2). The ROCK inhibitor Y-27632 also significantly attenuated the FRET signal for VEGFR2 tyrosine kinase. Consistent with the biosensor DMR indexes is that all 12 compounds shown in FIGS. 1 and 2 significantly inhibited the FRET signal of EGFR tyrosine kinase (FIG. 1), as well as VEGFR1 and VEGFR2 tyrosine kinases (FIG. 2 and FIG. 3, respectively). Compounds H and J and K were less potent than the others.

Cell proliferation assays using both HT29 and MCF7 under 2-dimensional culture conditions showed that all 12 compounds at 10 micromolar did not lead to significant cell apoptosis (data not shown). This is probably due to the low potency of these dual EGFR and VEGFR inhibitors, and/or 2-dimensional cell culture proliferation assays may not be good model for screening dual EGFR and VEGFR inhibitors, since the function of VEGFR inhibitors is better evident in 3-dimensional cell clusters.

### J. References

1. U.S. application Ser. No. 12/623,693. Fang, Y., Ferrie, A. M., Lahiri, J., and Tran, E. “Methods for Characterizing Molecules”, Filed Nov. 23, 2009
2. U.S. application Ser. No. 12/623,708. Fang, Y., Ferrie, A. M., Lahiri, J., and Tran, E. “Methods of creating an index”, filed Nov. 23, 2009.
3. Calvani, M. et al. “Differential involvement of vascular endothelial growth factor in the survival of hypoxic colon cancer cells”. Cancer Res. 2008, 68: 285
4. US20070265418. He, M. “Fused thiophenes, methods for making fused thiophenes, and uses thereof”.
5. US20070161776. He, M. “Fused thiophenes, methods for making fused thiophenes, and uses thereof”.

1. A method of inhibiting EGFR and VEGFR, comprising administering to a subject a compound or a pharmaceutically acceptable salt, solvate, clathrate, or prodrug thereof having the formula: 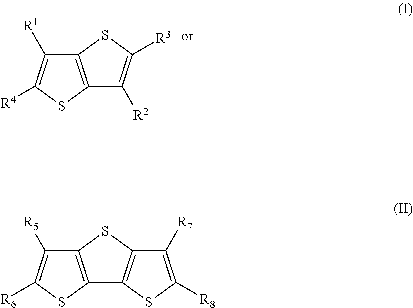
wherein R¹, R², R⁴, R⁵, R⁶ and R⁷ are independently —H, C₁-C₂₀ alkyl, C₁-C₂₀ alkynyl, C₁-C₂₀ alkenyl, aryl, alkylaryl, cycloalkyl, cycloalkenyl, heterocycyl, cyclohexyl, amino, ester, C₁-C₂₀ aldehyde, hydroxyl, C₁-C₂₀ alkoxy, thiol group, C₁-C₂₀ thioalkyl group, halogen, halide, or an acyl halide;
wherein R³ and R⁸ independently are —COOH, aldehyde or ester;
wherein the compound is a dual EGFR and VEGFR inhibitor.

2. The method of claim 1, wherein R³ and R⁸ are —COOH.

3. The method of claim 1, wherein R⁴ and R⁶ are —H.

4. The method of claim 3, wherein R¹ and R⁵ are independently halogen, —H, C₁-C₁₂ alkyl.

5. The method of claim 3, wherein R¹ and R⁵ are independently Br, —H, C₁-C₁₀ alkyl.

6. The method of claim 3, wherein R² and R⁷ are independently —H, C₁-C₂₀ alkyl.

7. The method of claim 3, wherein R² and R⁷ are independently —H, C₁, C₆, C₈, C₁₀, C₁₁, C₁₃ or C₁₅ alkyl.

8. The method of claim 1, wherein the compound or a pharmaceutically acceptable salt, solvate, clathrate, or prodrug thereof has the formula: 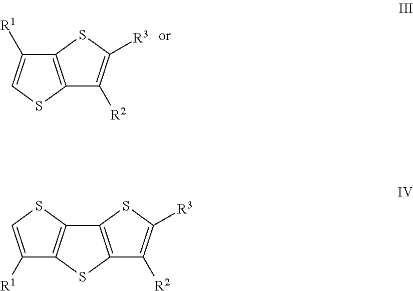
wherein:
R¹ is halogen, hydrogen, or unsubstituted C₁₋₂₀ alkyl,
R² is hydrogen or unsubstituted C₁₋₂₀ alkyl;
R³ is carboxyl;
at least one of R¹ and R² is unsubstituted C₁₋₂₀ alkyl; and
the compound is a dual EGFR and VEGFR inhibitor.

9. The method of claim 8, wherein R¹ is bromide, hydrogen or unsubstituted C₁, C₆, C₁₀ alkyl.

10. The method of claim 8, wherein R² is —H, C₁, C₆, C₈, C₁₀, C₁₁, C₁₃ or C₁₅ alkyl.

11. The composition of claim 8, wherein the compound is chosen from:

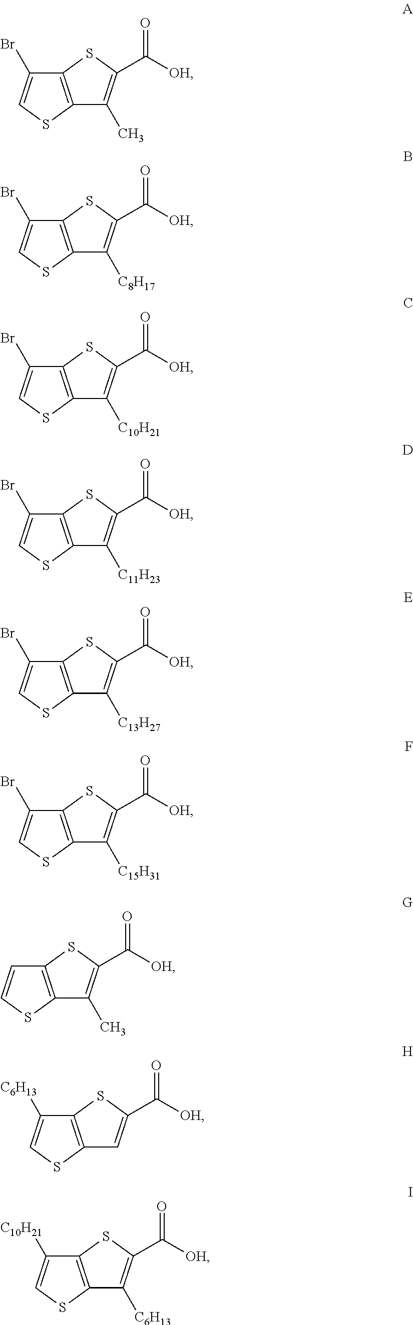

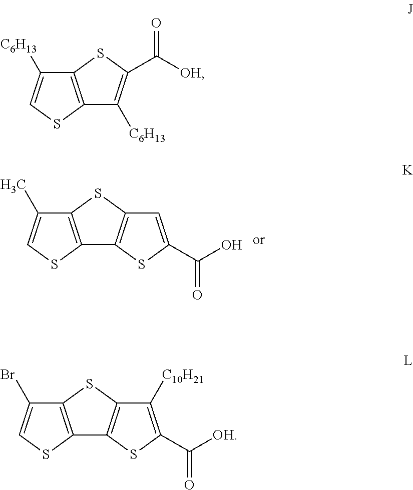

12. The method of claim 1, wherein the subject is in need of an EGFR and VEGFR inhibitor to treat or prevent a disease.

13. The method of claim 12, wherein the disease is cancer or a malignant disease.

14. The method of claim 13, wherein the cancer is skin cancer, colorectal cancer, breast cancer, thyroid cancer, non-small cell lung cancer, lung cancer, or pancreatic cancer.

15. A method to synthesize dual EGFR and VEGFR inhibitor, comprising linking a thieno[3,2-β]-thiophene scaffold with a building block that is a known fragment and functional group important for interactions with EGFR or VEGFR.

16. A method of screening dual EGFR and VEGFR inhibitors, comprising the steps:
a. selecting at least two distinct cell lines each expressing at least one receptor of EGFR or VEGFR,
b. selecting at least two markers, wherein one marker is the agonist for one of the two receptors, and another marker is an activator that transactivates another one of the two receptors,
c. incubating each marker in the absence and presence of a test compound to its respective cell line,
d. analyzing the biosensor signal of each marker in the absence and presence of a test compound on the marker respective cell line with a label free biosensor assay,
e. analyzing the effect of the test compound on all marker induced biosensor signals,
f. determining if the test compound is a dual EGFR and VEGFR inhibitor.

17. The method of claim 16, wherein the effect of the test compound is analyzed using a modulation index.

18. The method of claim 16, wherein the marker is selected from an agonist for EGFR, a G protein-coupled receptor agonist that transactivates EGFR, an agonist for VEGFR, or a hERG activator that transactivates VEGFR.

19. The method of claim 16, wherein the cell lines are A431 and HT29.

20. The method of claim 19, wherein the markers are selected from an EGFR agonist when the cell line is A431, and an EGFR agonist, a HERG activator and a GPCR agonist when the cell line is HT29.

21.-36. (canceled)
